(19) United States
(12) Patent Application Publication
(10) Pub. No.: US 2024/0156692 A1
(43) Pub. Date: May 16, 2024

(54) OIL-IN-WATER COMPOSITION FOR SECOND AGENT FOR COATING-TYPE BODY CORRECTIVE FILM FORMATION AGENT

(71) Applicant: SHISEIDO COMPANY, LTD., Chuo-ku, Tokyo (JP)

(72) Inventors: Satoshi YAMAKI, East Windsor, NJ (US); Melaney BOUTHILLETTE, East Windsor, NJ (US); Nithin RAMADURAI, East Windsor, NJ (US); Ariya AKTHAKUL, East Windsor, NJ (US)

(73) Assignee: SHISEIDO COMPANY, LTD., Chuo-ku, Tokyo (JP)

(21) Appl. No.: 18/280,820

(22) PCT Filed: Mar. 23, 2022
(86) PCT No.: PCT/JP2022/013695

§ 371 Date: Sep. 7, 2023

Related U.S. Application Data
(60) Provisional application No. 63/171,787, filed on Apr. 7, 2021.

(51) Int. Cl. A61K 8/06; A61Q 19/08

(52) U.S. Cl. CPC A61K 8/062; A61Q 19/08; A61K 2800/884; A61K 2800/95

(57) ABSTRACT

To provide a second agent for a coating-type body corrective film formation agent that can shorten the cross-linking reaction time in film formation and improve the durability of the film to be obtained.

An oil-in-water composition of the present disclosure comprises a dispersion medium containing water, and oil droplets dispersed in this dispersion medium, wherein the oil droplets comprise an oil, and a catalyst that serves as a cross-linking ingredient, wherein the oil-in-water composition is used as a second agent for a coating-type body corrective film formation agent comprising a first agent comprising a cross-linking reactive ingredient that constitutes a body corrective film; and a second agent comprising a cross-linking ingredient for cross-linking the cross-linking reactive ingredient.

### FIELD

The present disclosure relates to an oil-in-water composition for a second agent for a coating-type body corrective film formation agent.

### BACKGROUND

Coating-type film formation agents that can be applied to the body surface to form a film that can correct wrinkles, scars, and the like while protecting the skin are known.

Patent Literature 1 discloses a formulation for application to a skin, comprising: a) a reactive reinforcing ingredient containing (i) a reactive component containing at least one high-viscosity vinyl-terminated organopolysiloxane having a viscosity of 100,000 to 500,000 cSt or cP at 25° C., at least one low-viscosity vinyl-terminated organopolysiloxane having a viscosity of 500 to 50,000 cSt or cP at 25° C., and at least one hydride functionalized polysiloxane; and (ii) a reinforcing component; and b) a cross-linking ingredient containing a platinum catalyst; wherein the cross-linking ingredient promotes cross-linking of the reactive reinforcing ingredient in situ, resulting in formation of a film on the skin.

### CITATION LIST

### Patent Literature

[PTL 1] Japanese Patent No. 6105468

### SUMMARY

### Technical Problem

In the technology described in Patent Literature 1, a first agent containing a reactive component such as vinyl-terminated organopolysiloxane is applied to the skin to form a first agent layer, and then a second agent containing a platinum catalyst is applied to the first agent layer, followed by cross-linking the reactive component in the first agent layer to form a film on the skin.

Since the formation of the film by such a technology is achieved via the cross-linking reaction between the cross-linking reactive component in the first agent applied to the skin and the catalyst in the second agent, a certain length of cross-linking reaction time is required. A long cross-linking reaction time would cause the user to feel stress during the preparation of the film, or to touch the film that has not yet been cross-linked, which may result in failures such as tearing of the film. Thus, it has been desired to shorten the cross-linking reaction time in such a technology.

It has also been desired to further improve the durability of the film because a film formed by such a technology will be typically located on the top surface of the body and exposed to the external environment.

Accordingly, a subject matter of the present disclosure is to provide a second agent for a coating-type body corrective film formation agent that can shorten the cross-linking reaction time in film formation and improve the durability of the film to be obtained.

### Solution to Problem

### <Aspect 1>

An oil-in-water composition comprising:

a dispersion medium containing water, and

oil droplets dispersed in the dispersion medium,

wherein the oil droplets comprise an oil, and a catalyst that serves as a cross-linking ingredient,

wherein the oil-in-water composition is used as a second agent for a coating-type body corrective film formation agent comprising: a first agent comprising a cross-linking reactive ingredient that constitutes a body corrective film; and a second agent comprising a cross-linking ingredient for cross-linking the cross-linking reactive ingredient.

### <Aspect 2>

The composition according to Aspect 1, wherein the oil comprises a first unsaturated organopolysiloxane or a first hydride functionalized polysiloxane.

### <Aspect 3>

The composition according to Aspect 1 or 2, wherein the blending ratio of the catalyst to the oil is 0.060% or more, provided that, when the oil contains the first unsaturated organopolysiloxane or the first hydride functionalized polysiloxane, the blending ratio is calculated for the oil excluding the first unsaturated organopolysiloxane and the first hydride functionalized polysiloxane.

### <Aspect 4>

The composition according to any one of Aspects 1 to 3, comprising at least one emulsifier selected from the group consisting of a hydrocarbon surfactant, a silicone surfactant, and an amphiphilic powder.

### <Aspect 5>

The composition according to any one of Aspects 1 to 3, comprising at least one emulsifier selected from the group consisting of a polyoxyethylene alkyl ether, a polyoxyethylene steryl ether, a polyoxyethylene fatty acid ester, a polyoxyethylene polyhydric alcohol fatty acid ester, a polyoxyethylene hydrogenated castor oil, a polyoxyethylene sorbitan fatty acid ester, a glycol fatty acid ester, a glycerol fatty acid ester, a sorbitan fatty acid ester, a sucrose fatty acid ester, a polyglycerol fatty acid ester, a polyether-modified silicone, an alkyl co-modified polyether-modified silicone, and an amphiphilic powder.

### <Aspect 6>

The composition according to Aspect 4 or 5, wherein the blending amount of the emulsifier is 5% by mass or less with respect to the total amount of the oil-in-water composition.

### <Aspect 7>

The composition according to any one of Aspects 1 to 6, comprising a high-molecular-weight emulsifier.

### <Aspect 8>

The composition according to Aspect 7, wherein the high-molecular-weight emulsifier is at least one selected from the group consisting of an acrylates/C10-30 alkyl acrylate crosspolymer, an ammonium acryloyldimethyltaurate/beheneth-25 methacrylate crosspolymer, a hydroxyethyl acrylate/sodium acryloyldimethyl taurate copolymer, a PEG modified crosspolymer/copolymer siloxane, a polyether modified crosspolymer/copolymer siloxane, stearoxy hydroxypropyl methylcellulose, and polyoxyethylene.

### <Aspect 9>

The composition according to Aspect 7 or 8, wherein the blending amount of the high-molecular-weight emulsifier is 2% by mass or less with respect to the total amount of the oil-in-water composition.

### <Aspect 10>

The composition according to any one of Aspects 1 to 9, wherein the catalyst is at least one selected from the group consisting of a platinum carbonyl cyclovinylmethylsiloxane complex, a platinum divinyltetramethyldisiloxane complex, a platinum cyclovinylmethylsiloxane complex, and a platinum octanaldehyde/octanol complex.

### <Aspect 11>

A coating-type body corrective film formation agent comprising a first agent and a second agent,

wherein the first agent comprises at least one selected from the group consisting of a second unsaturated organopolysiloxane and a second hydride functionalized polysiloxane,

wherein the second agent is an oil-in-water composition according to any of Aspects 1 to 10,

wherein when the first agent only comprises the second unsaturated organopolysiloxane out of the second unsaturated organopolysiloxane and the second hydride functionalized polysiloxane, the second agent comprises the first hydride functionalized polysiloxane, and

wherein when the first agent only comprises the second hydride functionalized polysiloxane out of the second unsaturated organopolysiloxane and the second hydride functionalized polysiloxane, the second agent comprises the first unsaturated organopolysiloxane.

### <Aspect 12>

The formation agent according to Aspect 11, wherein the first unsaturated organopolysiloxane and the second unsaturated organopolysiloxane are at least one selected from the group consisting of organopolysiloxanes containing a vinyl group, vinyl-terminated organopolysiloxanes, and organopolysiloxanes containing a vinylated branched chain.

### <Aspect 13>

The formation agent according to Aspect 12, wherein the first unsaturated organopolysiloxane and the second unsaturated organopolysiloxane are at least one selected from the group consisting of vinyl-terminated polydimethylsiloxanes, vinyl-terminated diphenylsiloxane-dimethylsiloxane copolymers, vinyl-terminated polyphenylmethylsiloxanes, vinylphenylmethyl-terminated vinylphenylsiloxane-phenylmethylsiloxane copolymers, vinyl-terminated trifluoropropylmethylsiloxane-dimethylsiloxane copolymers, vinyl-terminated diethylsiloxane-dimethylsiloxane copolymers, vinylmethylsiloxane-dimethylsiloxane copolymers, trimethylsiloxy-terminated vinylmethylsiloxane-dimethylsiloxane copolymers, silanol-terminated vinylmethylsiloxane-dimethylsiloxane copolymers, vinylmethylsiloxane homopolymers, vinyl T-structure polymers, vinyl Q-structure polymers, monovinyl-terminated polydimethylsiloxanes, vinylmethylsiloxane terpolymers, and vinylmethoxysilane homopolymers.

### <Aspect 14>

The formation agent according to any one of Aspects 11 to 13, wherein the first hydride functionalized polysiloxane and the second hydride functionalized polysiloxane are non-terminally and/or terminally hydroxylated organopolysiloxanes.

### <Aspect 15>

The formation agent according to Aspect 14, wherein the first hydride functionalized polysiloxane and the second hydride functionalized polysiloxane are at least one selected from the group consisting of hydride-terminated polydimethylsiloxanes, hydride-terminated polyphenyl-(dimethylhydrosiloxy)siloxanes, hydride-terminated methylhydrosiloxane-phenylmethylsiloxane copolymers, trimethylsiloxy-terminated methylhydrosiloxane-dimethylsiloxane copolymers, polymethylhydrosiloxanes, trimethylsiloxy-terminated polyethylhydrosiloxanes, triethylsiloxanes, methylhydrosiloxane-phenyloctylmethylsiloxane copolymers, and methylhydrosiloxane-phenyloctylmethylsiloxane terpolymers.

### <Aspect 16>

The formation agent according to any one of Aspects 11 to 15, wherein at least one of the first agent and the second agent comprises at least one selected from the group consisting of a fiber, a pigment, a dye, a thickener, a UV absorber, and a filler.

### <Aspect 17>

A kit comprising the first agent and the second agent according to any one of Aspects 11 to 16, wherein the first agent and the second agent are contained in separate containers, or contained in separate compartments of a container having two or more compartments.

### <Aspect 18>

A method of using the formation agent according to any one of Aspects 11 to 16, comprising:

applying the first agent to the surface of a body to form a first agent layer, and then applying the second agent on the first agent layer to allow cross-linking, to form a body corrective film;

applying the second agent to the surface of a body to form a second agent layer, and then applying the first agent on the second agent layer to allow cross-linking, to form a body corrective film; or

mixing the first agent and the second agent to prepare a mixture, and then applying the mixture to the surface of a body to allow cross-linking, to form a body corrective film.

### <Aspect 19>

The method of use according to Aspect 18, comprising:

applying a cosmetic to the surface of the body before the application of the first agent, the second agent, or the mixture to the surface of the body;

applying the first agent to the surface of the body to form the first agent layer, applying a cosmetic to the first agent layer, and then applying the second agent to cover the cosmetic;

applying the second agent to the surface of the body to form the second agent layer, applying a cosmetic to the second agent layer, and then applying the first agent to cover the cosmetic; or

forming the body corrective film, and then applying a cosmetic to the film.

### <Aspect 20>

The method of use according to Aspect 19, wherein the cosmetic is a skin care cosmetic, a sunscreen cosmetic, a base cosmetic, or a make-up cosmetic, or is a cosmetic having a combination of two or more functions of these cosmetics.

### Advantageous Effects of Invention

The present disclosure can provide a second agent for a coating-type body corrective film formation agent, which second agent can shorten the cross-linking reaction time in the film formation and improve the durability of the resulting film.

### BRIEF DESCRIPTION OF DRAWINGS

FIG. 1(a) is a schematic view showing a state immediately after application of an oil-in-water composition of one embodiment of the present disclosure to a first agent layer, and FIG. 1(b) is a schematic view showing a state where oil droplets in an oil-in-water composition have moved to the surface of the first agent layer.

FIG. 2(a) is a schematic view showing a state immediately after application of an oil single-phase composition to a first agent layer, and FIG. 2(b) is a schematic view showing a state where a catalyst in the oil single-phase composition has moved to the surface of the first agent layer.

FIG. 3(a) is a schematic view showing a state immediately after application of a water-in-oil composition to a first agent layer, and FIG. 3(b) is a schematic view showing a state where a catalyst in the water-in-oil composition has moved to the surface of the first agent layer.

### DESCRIPTION OF EMBODIMENTS

In the following, embodiments of the present disclosure will be described in detail. The present disclosure is not limited to the following embodiments, and can be implemented with a variety of variations within the scope of the main purpose of the invention.

The oil-in-water composition of the present disclosure comprises: a dispersion medium containing water; and oil droplets dispersed in the dispersion medium; wherein the oil droplets comprise an oil, and a catalyst that serves as a cross-linking ingredient. The oil-in-water composition is used as a second agent for a coating-type body corrective film formation agent comprising: a first agent comprising a cross-linking reactive ingredient that constitutes a body corrective film; and a second agent comprising a cross-linking ingredient for cross-linking the cross-linking reactive ingredient.

Without being limited by any principle, the following is thought to be a principle of action by which the oil-in-water composition of the present disclosure enables shortening of the cross-linking reaction time in the film formation and improvement of the durability of the resulting film when the composition is used as a second agent for a coating-type body corrective film formation agent.

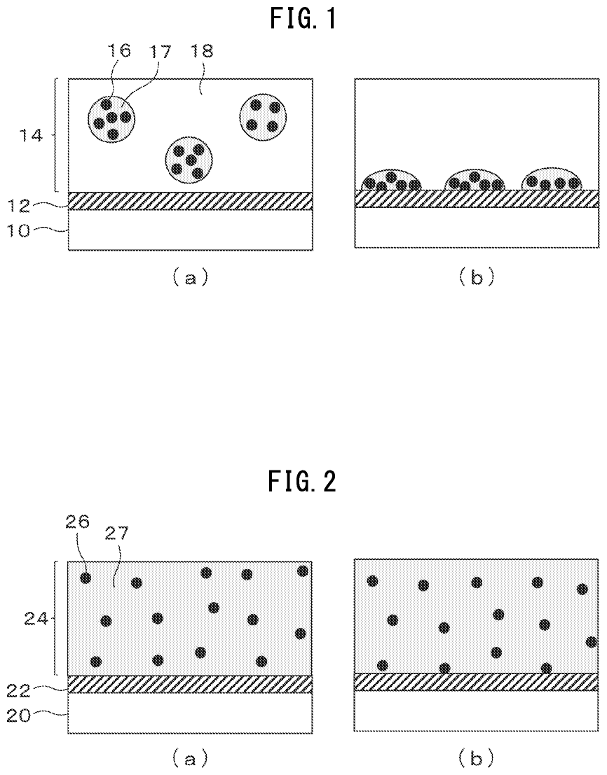

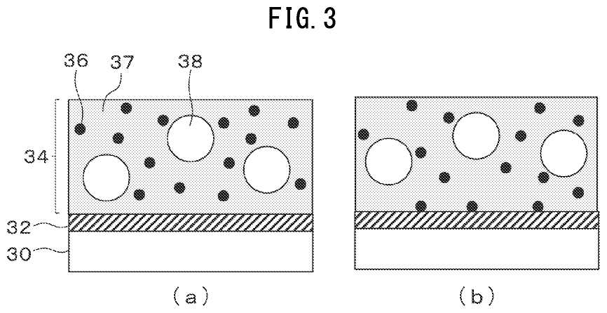

The catalyst as a cross-linking ingredient in the second agent is typically contained in the oil. Examples of such a form in which the catalyst is contained in the oil can include the form as an oil-in-water composition shown in FIG. 1(a), the form as an oil single phase shown in FIG. 2(a), and the form as a water-in-oil composition shown in FIG. 3(a).

In the formation of the coating-type body corrective film (which may be referred to as “film”) in the present disclosure, for example, a layer containing unsaturated organopolysiloxane and hydride functionalized polysiloxane as cross-linking reactive ingredients that constitute the body corrective film is brought into contact with the catalyst, to thereby allow the cross-linking reaction to proceed between the unsaturated organopolysiloxane and the hydride functionalized polysiloxane.

The present inventor has found that the contact with the catalyst occurs differently depending on the formulation of the second agent, and that such a difference affects the cross-linking reaction time in the film formation and leads to a difference in the durability of the resulting film. The influence on the cross-linking reaction time in the film formation, and the film durability, will be described in detail below with reference to FIGS. 1 to 3.

For example, as shown in FIG. 1(a), when the first agent is applied to the skin, a first agent layer 12 is formed. When the second agent is applied to the first agent layer 12 to form a second agent layer 14, the oil droplets in the second agent layer immediately after the formation of the second agent layer 14 are thought to be dispersed in the second agent layer. The first agent layer 12 containing unsaturated organopolysiloxane and/or hydride functionalized polysiloxane is lipophilic, and thus has better affinity with the oil 17 than with the aqueous phase 18. Therefore, the oil droplets in the second agent layer are more likely to move to the surface of the first agent layer and spread out to blend as shown in FIG. 1(b), rather than to disperse in the second agent layer. It is thought that, as a result, the catalyst 16 in the oil droplets is more likely to contact the first agent layer 12.

On the other hand, in cases where the second agent is in the form of an oil single phase or a water-in-oil type, application of the second agent to the first agent layer 22 or 32 as shown in FIGS. 2(b) and 3(b) is thought to hardly cause movement of the catalyst 26 or 36 dispersed in the oil 27 or 37 to the first agent layer 22 or 32, unlike the cases of the oil-in-water type. As a result, most of the catalyst 26 or 36 in the second agent layer 24 or 34 is thought to remain dispersed in the second agent layer 24 or 34, i.e., not contributing to the cross-linking reaction.

It is thus thought that an oil-in-water formulation can shorten the cross-linking reaction time in the film formation because it allows the catalyst as the cross-linking ingredient to move more easily to the surface of the first agent layer as compared to an oil single-phase formulation or a water-in-oil formulation. In addition, an oil-in-water formulation is thought to allow better use of the catalyst blended in the second agent during the cross-linking reaction, leading to a favorable increase in the cross-linking density of the film, which results in an improved durability of the resulting film.

The definitions of terms in the present disclosure are as follows.

“Body corrective film” in the present disclosure means a film that is intended to exhibit a natural appearance of a skin when formed on the skin of a subject. Herein, the term “natural appearance of a skin” means that the body corrective film, when applied to the skin, exhibits a performance similar or identical to at least one selected from the external appearance, feel, and texture of the actual skin. For example, the term means that the skin subjected to the film treatment can exhibit physical properties (e.g., elasticity and firmness) of the actual (e.g., current) skin.

The term “body correction” in the present disclosure means that a body defect or skin defect of a subject is masked, hidden, or covered to visually and/or tactilely improve the body or skin defect, but the meaning does not include a method of operating, treating, or diagnosing a human. Herein, the term “body defect” may mean, for example, an area of a subject's body that the subject perceives as a blemish or scar, or that a person skilled in the art, e.g., a dermatologist, an esthetician, or an orthopedist, considers to be a blemish or scar. The term “body defect” includes skin defects and soft tissue laxity of the body (e.g., looseness or laxity of the skin; and laxity of the breasts, buttocks, abdomen, chin, neck, and the like). The term “skin defect” includes those items of the subject's skin that the subject perceives as blemishes or scars. Examples of the skin defect include nevus flammeus or flame nevus (e.g., simple hemangioma or median flamme nevus), chloasmas, wrinkles, blemishes, acnes, moles, scars, tattoos, birthmarks, skin deformation, nevi, suntans, aging, uneven skin tones, lax skins, rough skins, hyperpigmentation, enlarged pores, telangiectasia, redness, shine, cellulite, striae gravidarum, and reduced skin elasticity.

As used herein, the term “oil-in-water composition” is intended to mean a composition that is in a state where oil droplets are dispersed in a dispersion medium containing water. Examples of such a composition may include a composition prepared by shaking a liquid that is in a state showing separation into water and an oil, to forcibly disperse oil droplets in a dispersion medium containing the water, and an emulsion composition prepared by blending an emulsifier to disperse oil droplets in a dispersion medium containing water.

In the present disclosure, “viscosity” refers to a measure of resistance of a fluid being deformed by either shear stress or tensile stress. For example, the viscosities of the first agent and the second agent in the coating-type body corrective film formation agent have effects on the thickness, the spreadability, and the uniformity and/or the evenness of a layer formed on a substrate. A viscosity can be reported as either a dynamic viscosity (also known as absolute viscosity; typically in the unit of Pa·s, Poise, P, or cP) or a kinematic viscosity (typically in the unit of cm²/s, Stokes, St, or cSt), and this kinematic viscosity is obtained by dividing the dynamic viscosity by the measured density of the fluid. The viscosity range of an ingredient disclosed herein is generally provided by the supplier of each ingredient in a unit of kinematic viscosity (for example, cSt) based on measurement using a rheometer or a Cannon-Fenske tube viscometer, and the viscosity of a fluid can also be measured using, for example, a rheometer (for example, linear shear rheometer or dynamic shear rheometer) or a viscometer (also referred to as viscometric apparatus, for example, capillary viscometer or rotational viscometer).

The term “cross-linking” herein also encompasses the concept generally referred to as “curing”.

In the present disclosure, “body surface” means the skin surface of a body.

«Oil-in-water Composition»

The oil-in-water composition (which may be referred to as simply “composition”) of the present disclosure can be used as a second agent for a coating-type body corrective film formation agent comprising: a first agent containing a cross-linking reactive ingredient that constitutes the body corrective film; and a second agent containing a cross-linking ingredient for cross-linking the cross-linking reactive ingredients; and can shorten the cross-linking reaction time in the film formation. For example, the cross-linking reaction time can be within 2 minutes, within 1 minute 30 seconds, within 1 minute, within 50 seconds, or within 40 seconds in a case where a second agent containing a catalyst is applied to a first agent layer containing unsaturated organopolysiloxane and hydride functionalized polysiloxane as cross-linking reactive ingredients that constitute the body corrective film. The lower limit of such a cross-linking reaction time is not particularly limited, and can be, for example, 1 second or more, 5 seconds or more, or 10 seconds or more. Herein, the term “cross-linking reaction time” is intended to mean the time period from the point when the catalyst comes into contact with the unsaturated organopolysiloxane and the hydride functionalized polysiloxane to the point when curing of the surface of the film formed by the body corrective film formation agent has proceeded to a level at which no film damage occurs even by touching with a finger.

The oil-in-water composition of the present disclosure can improve the durability of the film to be obtained. Such durability can be evaluated based on the presence or absence of defects such as peeling or damage of the film immediately after the formation. In visual observation, the film prepared using the oil-in-water composition of the present disclosure can exhibit a condition showing slight peeling and/or damage, or showing neither peeling nor damage in the film.

The durability of the obtained film can also be evaluated by the abrasion resistance test described later. The film prepared using the oil-in-water composition of the present disclosure can achieve a number of repetition, until occurrence of damage in the film in the abrasion resistance test, of 5 times or more, 7 times or more, 10 times or more, 12 times or more, 15 times or more, 17 times or more, or 20 times or more. The upper limit of such a number of repetition is not particularly limited, and can be, for example, 200 times or less, 150 times or less, or 100 times or less.

In some embodiments, the oil-in-water composition of the present disclosure has excellent emulsifiability. The emulsifiability can be evaluated based on the uniformity of the emulsion particles (oil droplets) in the oil-in-water composition, i.e., the emulsion particle size variation. The oil-in-water composition of the present disclosure can have an emulsion particle (oil droplet) size variation of 10 μm or less, 8 μm or less, or 5 μm or less. The lower limit of such a variation is not particularly limited, and can be, for example, no variation (0 μm), more than 0 μm, 0.5 μm or more, or 1 μm or more. Here, the emulsion particle size is a value obtained by observation with a light microscope.

<Dispersion Medium>

The dispersion medium in the oil-in-water composition of the present disclosure contains water.

(Water)

The blending amount of the water is not particularly limited, and can be, for example, 15% by mass or more, 20% by mass or more, 30% by mass or more, 40% by mass or more, 50% by mass or more, 60% by mass or more, 70% by mass or more, or 80% by mass or more, and can be, for example, 90% by mass or less, 80% by mass or less, 70% by mass or less, 60% by mass or less, or 50% by mass or less, with respect to the total amount of the composition from the viewpoints of usability, cross-linking reactivity, and the like.

Water that can be used in the oil-in-water composition of the present disclosure is not particularly limited. For example, water that is used in cosmetics or quasi-drugs can be used. For example, ion exchanged water, distilled water, ultrapure water, or tap water can be used.

<Oil Droplets>

The oil droplets as the oil phase or dispersed phase in the oil-in-water composition contains an oil, and a catalyst as a cross-linking ingredient.

(Oil)

The blending amount of the oil is not particularly limited, and can be, for example, 0.01% by mass or more, 0.03% by mass or more, 0.05% by mass or more, 0.07% by mass or more, 0.1% by mass or more, 0.3% by mass or more, 0.5% by mass or more, 0.7% by mass or more, 1% by mass or more, 3% by mass or more, 5% by mass or more, 7% by mass or more, 10% by mass or more, 15% by mass or more, or 20% by mass or more, and can be, for example, 50% by mass or less, 40% by mass or less, 30% by mass or less, 20% by mass or less, 15% by mass or less, or 10% by mass or less, with respect to the total amount of the composition from the viewpoints of usability, cross-linking reactivity, and the like.

The oil is not particularly limited, and examples thereof include liquid oils and fats, solid oils and fats, waxes, hydrocarbon oils, silicone oils, and polar oils. The oils can be used singly or in combination of two or more kinds thereof. Among them, a silicone oil is preferred from the viewpoints of affinity with the first agent layer formed by the first agent as described later, and the like. The ratio of the silicone oil in the oil is not particularly limited, and can be, for example, 50% by mass or more, 60% by mass or more, 70% by mass or more, 80% by mass or more, or 90% by mass or more with respect to the total amount of the oil. The upper limit of such a ratio is not particularly limited, and can be, for example, 100% by mass or less, less than 100% by mass, 98% by mass or less, or 95% by mass or less.

a. First Unsaturated Organopolysiloxane

The composition of the present disclosure can contain, as an oil, a first unsaturated organopolysiloxane that is one of silicone oils. The first unsaturated organopolysiloxane can function as a component of the film, and can also function as a dispersant for a catalyst.

The first unsaturated organopolysiloxane is not particularly limited, and may be, for example, one or more organopolysiloxanes having at least two carbon-carbon double bonds or at least one carbon-carbon triple bond in the molecule. Preferred examples of such an unsaturated organopolysiloxane include one or more organopolysiloxanes containing at least two alkenyl functional groups on average and having a viscosity of from 10,000 to 2,000,000 cSt at 25° C. Herein, “carbon-carbon double bond” and “carbon-carbon triple bond” may be referred to simply as “double bond” and “triple bond,” respectively. The first unsaturated organopolysiloxanes can be used singly or in combination of two or more kinds thereof.

Such an organopolysiloxane may contain a double bond or a triple bond in a terminal unit of the polymer, in a non-terminal monomer unit of the polymer, or in a combination thereof, particularly preferably in a non-terminal monomer unit of the polymer.

In one embodiment, double bond-containing monomer units in the organopolysiloxane may be apart from each other by, on average, 40 monomer units or more, 200 monomer units or more, 400 monomer units or more, 1,000 monomer units or more, or 2,000 monomer units or more.

In one embodiment, the amount of monomer units containing a double bond or a triple bond in the organopolysiloxane containing a double bond or a triple bond can be, for example, 0.01% by mass or more or 0.03% by mass or more, and can be 2% by mass or less or 0.6% by mass or less.

In one embodiment, the vinyl equivalent of the organopolysiloxane containing a double bond or a triple bond can be, for example, 0.005 or more or 0.01 or more per kilogram, and can be 0.5 or less or 0.25 or less per kilogram. The approximate molar amount of double bonds or triple bonds in the organopolysiloxane can be calculated based on the average molecular weight of the organopolysiloxane. The average molecular weight or molecular mass of each ingredient disclosed herein is generally provided by the supplier of each ingredient and can be expressed in daltons (Da) or in a unit equivalent thereto, g/mol.

In one embodiment, the first unsaturated organopolysiloxane can have a viscosity of from 10,000 to 2,000,000 cSt at 25° C. The lower limit of the viscosity is preferably 20,000 cSt or more, 40,000 cSt or more, 60,000 cSt or more, 80,000 cSt or more, or 100,000 cSt or more, and more preferably 125,000 cSt or more or 150,000 cSt or more. The upper limit of the viscosity is preferably 1,000,000 cSt or less, 500,000 cSt or less, 450,000 cSt or less, 400,000 cSt or less, 350,000 cSt or less, 300,000 cSt or less, or 250,000 cSt or less, more preferably 200,000 cSt or less or 180,000 cSt or less, and still more preferably 165,000 cSt or less.

In one embodiment, the first unsaturated organopolysiloxane can have an average molecular weight of from 60,000 Da to 500,000 Da. The lower limit of such an average molecular weight is preferably 72,000 Da or more, 84,000 Da or more, 96,000 Da or more, or 100,000 Da or more, and more preferably 140,000 Da or more or 150,000 Da or more. The upper limit of the average molecular weight is preferably 200,000 Da or less, 190,000 Da or less, 180,000 Da, or 170,000 Da or less, more preferably 160,000 Da or less, and still more preferably 155,000 Da or less.

Examples of the first unsaturated organopolysiloxane that can be used include at least one unsaturated organopolysiloxane selected from the group consisting of organopolysiloxanes containing a vinyl group, vinyl-terminated organopolysiloxanes, and organopolysiloxanes containing a vinylated branched chain.

Specific examples of the first unsaturated organopolysiloxane include vinyl-terminated polydimethylsiloxane, vinyl-terminated diphenylsiloxane-dimethylsiloxane copolymer, vinyl-terminated polyphenylmethylsiloxane, vinylphenylmethyl-terminated vinylphenylsiloxane-phenylmethylsiloxane copolymer, vinyl-terminated trifluoropropylmethylsiloxane-dimethylsiloxane copolymer, vinyl-terminated diethylsiloxane-dimethylsiloxane copolymer, vinylmethylsiloxane-dimethylsiloxane copolymer, trimethylsiloxy-terminated vinylmethylsiloxane-dimethylsiloxane copolymer, silanol-terminated vinylmethylsiloxane-dimethylsiloxane copolymer, vinylmethylsiloxane homopolymer, vinyl T-structure polymer, vinyl Q-structure polymer, monovinyl-terminated polydimethylsiloxane, vinylmethylsiloxane terpolymer, and vinylmethoxysilane homopolymer. The first unsaturated organopolysiloxane can be used singly or in combination of two or more kinds thereof. Among them, vinyl-terminated polydimethylsiloxane is preferred, and vinyl dimethicone (divinyl dimethicone) is more preferred. In this disclosure, “terminal” is intended to mean either one or both terminals. In order to distinguish between them, they can be denoted, for example, as “one vinyl terminal” and “both vinyl terminals”.

b. First Hydride Functionalized Polysiloxane

The composition of the present disclosure can contain, as an oil, a first hydride functionalized polysiloxane that is one of silicone oils. The first hydride functionalized polysiloxane can function as a component of the film.

The first hydride functionalized polysiloxane is not particularly limited, and examples thereof include a compound represented by the Formula 1 below. The first hydride functionalized polysiloxane can be used singly or in combination of two or more kinds thereof.

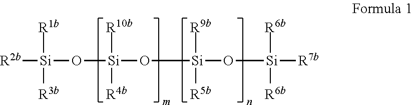

In Formula 1, R^{1b}, R^{2b}, R^{3b}, R^{4b}, R^{5b}, R^{6b}, R^{7b}, R^{8b}, R^{9b}, and R^{10b} are each independently selected from hydrogen, C₁₋₂₀ alkyl, C₂₋₂₀ alkenyl, C₅₋₁₀ aryl, hydroxyl, or C₁₋₂₀ alkoxy, and m and n are each independently an integer of 10 to 6,000, provided that at least one of R^{1b}, R^{2b}, R^{3b}, R^{4b}, R^{5b}, R^{6b}, R^{7b}, R^{8b}, R^{9b}, and R^{10b} is hydrogen.

In some embodiments, at least one of R^{1b}, R^{2b}, R^{3b}, R^{4b}, R^{5b}, R^{6b}, R^{7b}, R^{8b}, R^{9b}, and R^{10b} is hydrogen, and the rest is C₁₋₂₀ alkyl.

In some embodiments, at least two of R^{1b}, R^{2b}, R^{3b}, R^{4b}, R^{5b}, R^{6b}, R^{7b}, R^{8b}, R^{9b}, and R^{10b} are hydrogen (for example, two Si—H units per functionalized hydride polysiloxane molecule).

In other embodiments, at least three of R^{1b}, R^{2b}, R^{3b}, R^{4b}, R^{5b}, R^{6b}, R^{7b}, R^{8b}, R^{9b}, and R^{10b} are hydrogen (for example, three Si—H units per functionalized hydride polysiloxane molecule).

In some embodiments, at least two of R^{1b}, R^{2b}, R^{3b}, R^{4b}, R^{5b}, R^{6b}, R^{7b}, R^{8b}, R^{9b}, and R^{10b} are hydrogen (for example, two Si—H units per functionalized hydride polysiloxane molecule), and the rest is C₁₋₂₀ alkyl.

In other embodiments, at least three of R^{1b}, R^{2b}, R^{3b}, R^{4b}, R^{5b}, R^{6b}, R^{7b}, R^{8b}, R^{9b}, and R^{10b} are hydrogen (for example, three Si—H units per functionalized hydride polysiloxane molecule), and the rest is C₁₋₂₀ alkyl.

In some embodiments, at least two of R^{4b}, R^{5b}, R^{9b}, and R^{10b} are hydrogen (for example, two Si—H units per functionalized hydride polysiloxane molecule), and the rest is C₁₋₂₀ alkyl.

In other embodiments, at least three of R^{4b}, R^{5b}, R^{9b}, and R^{10b} are hydrogen (for example, three Si—H units per functionalized hydride polysiloxane molecule), and the rest is C₁₋₂₀ alkyl.

In some embodiments, the total of m and n is an integer of 10 to 1,300, 10 to 1,100, 10 to 600, 15 to 500, 15 to 400, 20 to 300, 20 to 200, 25 to 100, 25 to 75, 30 to 50, or 40 to 45.

In some embodiments, the first hydride functionalized polysiloxane may be non-terminally and/or terminally hydrogenated organopolysiloxane, and is composed of one or more organopolysiloxanes containing at least two Si—H units in the molecule. The first hydride functionalized polysiloxane may preferably be one or more organopolysiloxanes containing at least two Si—H units on average and having a viscosity of from 2 to 100,000 cSt at 25° C.

In one embodiment, the organopolysiloxane containing an Si—H unit may contain such an Si—H unit in a terminal unit of the polymer, in a non-terminal monomer unit of the polymer, or in a combination thereof. Among them, an Si—H unit is preferably contained in a non-terminal monomer unit of the polymer. In this case, the first hydride functionalized polysiloxane may be alkyl-terminated. For example, in Formula 1, one or both of R^{2b} and R^{7b} may be C₁₋₂₀ alkyl.

In one embodiment, in Formula 1, one, two, three, four, five, or six of R^{1b}, R^{2b}, R^{3b}, R^{6b}, R^{7b}, and R^{8b} may be C₁₋₂₀ alkyl.

In one embodiment, R^{1b}, R^{2b}, R^{3b}, R^{4b}, R^{5b}, R^{6b}, R^{7b}, R^{8b}, and R^{10b} each may be C₁₋₂₀ alkyl, such as C₁ alkyl (such as methyl), and R^{9b} may be hydrogen.

In one embodiment, R^{1b}, R^{2b}, R^{3b}, R^{4b}, R^{5b}, R^{6b}, R^{7b}, R^{8b}, and R^{9b} each may be C₁₋₂₀ alkyl, such as C₁ alkyl (such as methyl), and R^{10b} may be hydrogen.

In one embodiment, Si—H-containing monomer units in the organopolysiloxane may be apart from each other by 1 monomer unit or more, 2 monomer units or more, 5 monomer units or more, 10 monomer units or more, 20 monomer units or more, 40 monomer units or more, 200 monomer units or more, 400 monomer units or more, 1,000 monomer units or more, or 2,000 monomer units or more on average.

In one embodiment, the amount of monomer units containing an Si—H unit in the organopolysiloxane containing an Si—H unit can be 0.003% by mass or more, 0.01% by mass or more, 0.1% by mass or more, 1% by mass or more, 3% by mass or more, 5% by mass or more, 10% by mass or more, 20% by mass or more, or 26% by mass or more, and can be 50% by mass or less, 45% by mass or less, 40% by mass or less, 35% by mass or less, 30% by mass or less, or 27% by mass or less.

In one embodiment, the Si—H content in the organopolysiloxane containing an Si—H unit can be 0.1 mmol/g or more, 0.5 mmol/g or more, 1 mmol/g or more, 2 mmol/g or more, 3 mmol/g or more, or 4 mmol/g or more, and can be 20 mmol/g or less, 10 mmol/g or less, 9 mmol/g or less, 8 mmol/g or less, 7 mmol/g or less, 6 mmol/g or less, or 5 mmol/g or less. The approximate molar amount of Si—H units in the organopolysiloxane can be calculated based on the average molecular weight of the organopolysiloxane.

In one embodiment, the first hydride functionalized polysiloxane can have a viscosity of from 2 to 500,000 cSt at 25° C. The lower limit of such a viscosity is preferably 3 cSt or more, 4 cSt or more, 5 cSt or more, 10 cSt or more, 12 cSt or more, 15 cSt or more, 20 cSt or more, 25 cSt or more, or 30 cSt or more, and more preferably 40 cSt or more. The upper limit of the viscosity is preferably 200,000 cSt or less, 100,000 cSt or less, 50,000 cSt or less, 20,000 cSt or less, 10,000 cSt or less, 5,000 cSt or less, 2,000 cSt or less, or 1,000 cSt or less, and more preferably 500 cSt or less. The viscosity of the hydride functionalized polysiloxane is particularly preferably in the range of from 45 to 100 cSt or from 45 to 50 cSt at 25° C.

In one embodiment, the hydride functionalized polysiloxane can have an average molecular weight of from 400 to 500,000 Da. The lower limit of such an average molecular weight is preferably 500 Da or more, 800 Da or more, 900 Da or more, 1,000 Da or more, 1,200 Da or more, 1,400 Da or more, 1,600 Da or more, 1,800 Da or more, 2,000 Da or more, or 2,200 Da or more, and more preferably 2,300 Da or more. The upper limit of the average molecular weight is preferably 250,000 Da or less, 140,000 Da or less, 100,000 Da or less, 72,000 Da or less, 62,700 Da or less, 60,000 Da or less, 50,000 Da or less, 49,500 Da or less, 36,000 Da or less, 28,000 Da or less, 25,000 Da or less, 20,000 Da or less, 15,000 Da or less, 10,000 Da or less, 5,000 Da or less, or 4,000 Da or less, and more preferably 2,500 Da or less.

The first hydride functionalized polysiloxane that can be employed may be, for example, but not limited to, at least one selected from the group consisting of hydride-terminated poly dimethylsiloxane, hydride-terminated polyphenyl-(dimethylhydrosiloxy)siloxane, hydride-terminated methylhydrosiloxane-phenylmethylsiloxane copolymer, trimethylsiloxy-terminated methylhydrosiloxane-dimethylsiloxane copolymer, polymethylhydrosiloxane, trimethylsiloxy-terminated polyethylhydrosiloxane, triethylsiloxane, methylhydrosiloxane-phenyloctylmethylsiloxane copolymer, and methylhydrosiloxane-phenyloctylmethylsiloxane terpolymer. Among them, hydride-terminated poly dimethylsiloxane is preferred, and hydrogen dimethicone is more preferred.

c. Other Silicone Oils

Silicone oils other than the first unsaturated organopolysiloxane and the first hydride functionalized polysiloxane are not particularly limited, and examples thereof that can be used include chain silicones such as dimethylpolysiloxane (dimethicone), methylphenylpolysiloxane, and methylhydrogen polysiloxane; and cyclic silicones such as octamethyl cyclotetrasiloxane, decamethyl cyclopentasiloxane, and dodecamethyl cyclohexasiloxane. The other silicone oils can be used singly or in combination of two or more kinds thereof.

(Catalyst)

The catalyst is not particularly limited, and can be, for example, any substance capable of causing, promoting, or initiating a physical and/or chemical cross-linking reaction targeting the unsaturated organopolysiloxane and the hydride functionalized polysiloxane that are cross-linking reactive ingredients constituting the body corrective film. The catalyst optionally undergoes permanent physical and/or chemical changes during or at the end of the process.

The catalyst can include, but is not limited to, a metal catalyst capable of initiating and/or promoting cross-linking at or below body temperature, and examples thereof include a Group VIII metal catalyst such as a platinum catalyst, a rhodium catalyst, a palladium catalyst, a cobalt catalyst, a nickel catalyst, a ruthenium catalyst, an osmium catalyst, or an iridium catalyst, and a Group IVA metal catalyst such as a germanium catalyst or a tin catalyst. Among them, a platinum catalyst, a rhodium catalyst, or a tin catalyst is preferred. The catalysts can be used singly or in combination of two or more kinds thereof.

Examples of the platinum catalyst include a platinum carbonyl cyclovinylmethylsiloxane complex, a platinum divinyltetramethyldisiloxane complex, a platinum cyclovinylmethylsiloxane complex, a platinum octanaldehyde/octanol complex, and another Pt(0) catalyst, such as a Karstedt's catalyst, a platinum-alcohol complex, a platinum-alkoxide complex, a platinum-ether complex, a platinum-aldehyde complex, a platinum-ketone complex, a platinum-halogen complex, a platinum-sulfur complex, a platinum-nitrogen complex, a platinum-phosphorus complex, a platinum-carbon double-bond complex, a platinum-carbon triple-bond complex, a platinum-imide complex, a platinum-amide complex, a platinum-ester complex, a platinum-phosphate complex, a platinum-thiol ester complex, a platinum-isolated electron pair complex, a platinum-aromatic complex, a platinum-pi-electron complex, and a combination thereof. Among them, at least one selected from the group consisting of a platinum carbonyl cyclovinylmethylsiloxane complex, a platinum divinyltetramethyldisiloxane complex, a platinum cyclovinylmethylsiloxane complex, and a platinum octanaldehyde/octanol complex is preferred.

Examples of the rhodium catalyst include tris(dibutylsulfide)rhodium trichloride and rhodium trichloride hydrate.

Examples of the tin catalyst include tin (II) octanoate, tin (II) neodecanoate, dibutyltin diisooctylmaleate, di-n-butylbis(2,4-pentanedionate)tin, di-n-butylbutoxychlorotin, dibutyltin dilaurate, dimethyltin dineodecanoate, dimethylhydroxy(oleate)tin, and tin (II) oleate.

Among these catalysts, a platinum catalyst is more preferred, and a platinum divinyltetramethyldisiloxane complex is particularly preferred.

The blending amount of the catalyst in the oil-in-water composition may be adjusted as appropriate according to the film performance or the like required, and is not particularly restricted. For example, the blending amount of the catalyst with respect to the total amount of the composition can be 0.001% by mass or more, 0.005% by mass or more, or 0.010% by mass or more, and can be 1.0% by mass or less, 0.10% by mass or less, or 0.050% by mass or less.

The blending ratio of the catalyst to the oil described above can be 0.060% or more, 0.10% or more, 0.50% or more, 1.0% or more, 5.0% or more, 10% or more, 15% or more, or 20% or more from the viewpoints of shortening the cross-linking reaction time, film durability, and the like. The upper limit of such a blending ratio is not particularly limited, and can be, for example, 90% or less, 80% or less, 70% or less, 60% or less, 50% or less, 40% or less, or 30% or less. Here, the blending ratio of the catalyst to the oil can be calculated according to Equation 2 below. It is noted that, when the oil contains the first unsaturated organopolysiloxane or the first hydride functionalized polysiloxane, the blending ratio is calculated for the oil excluding the first unsaturated organopolysiloxane and the first hydride functionalized polysiloxane that are reactive polymers:

Blending ratio (%)=(catalyst content (g)×100)/oil content (g) Equation 2

In some embodiments, when the oil-in-water composition of the present disclosure uses, for example, a platinum complex as the catalyst, the mass ratio of the first unsaturated organopolysiloxane to the total amount of platinum in the platinum complex may be less than 200.

(Emulsifier)

In some embodiments, the oil-in-water composition of the present disclosure can contain an emulsifier. The emulsifier in the present disclosure means an agent having an emulsifying function (surface-active properties), and can also include an agent generally called surfactant. An oil-in-water composition containing an emulsifier can be referred to as oil-in-water emulsion composition.

The blending amount of the emulsifier is not particularly limited, and can be, for example, 0.01% by mass or more, 0.05% by mass or more, 0.1% by mass or more, or 0.2% by mass or more with respect to the total amount of the composition from the viewpoint of emulsion stability and the like. The upper limit of the blending amount of the emulsifier is not particularly limited, and the blending amount of the emulsifier is preferably, for example, 5% by mass or less, 4% by mass or less, 3% by mass or less, 2% by mass or less, or 1% by mass or less from the viewpoint of film durability and the like.

As the emulsifier, for example, an anionic, cationic, amphoteric, or nonionic emulsifier can be used. The emulsifiers can be used singly or in combination of two or more kinds thereof.

Specific examples of the emulsifier include at least one selected from the group consisting of hydrocarbon surfactants, silicone surfactants, high-molecular-weight emulsifiers, and amphiphilic powders.

Examples of the hydrocarbon surfactants include polyoxyethylene alkyl ethers, polyoxyethylene steryl ethers, polyoxyethylene fatty acid esters, polyoxyethylene polyhydric alcohol fatty acid esters, polyoxyethylene hydrogenated castor oils, polyoxyethylene sorbitan fatty acid esters, glycol fatty acid esters, glycerol fatty acid esters, sorbitan fatty acid esters, sucrose fatty acid esters, and polyglycerol fatty acid esters.

Examples of the silicone surfactants include polyether-modified silicones, and alkyl co-modified polyether-modified silicones.

Among the emulsifiers, high-molecular-weight emulsifiers are preferred from the viewpoints of uniform dispersibility (emulsifiability) of the catalyst in the composition, storage stability of the catalyst, and the like. Typically, high-molecular-weight emulsifiers can mean emulsifiers (surfactants) that have larger molecular weights and lower emulsifying abilities than common emulsifiers (surfactants). A high-molecular-weight emulsifier may be used in combination with the emulsifier described above.

The weight average molecular weight of the high-molecular-weight emulsifier can be 500 or more, 700 or more, 1,000 or more, 1,500 or more, or 2,000 or more from the viewpoint of emulsifiability and the like. The upper limit of the weight average molecular weight of the high-molecular-weight emulsifier is not particularly limited, and can be, for example, 1,000,000 or less, 100,000 or less, 10,000 or less, or 5,000 or less. The weight average molecular weight of the emulsifier is a value in terms of polystyrene determined by preparing a 0.5% solution of the emulsifier dissolved in N,N-dimethylformamide (DMF), and using this solution to perform GPC (gel permeation chromatography) measurement under the following conditions:

Column: Two α-M (manufactured by Showa Denko K.K.) columns connected in tandem were used;

Eluent: A solution of 60 mmol/L H₃PO₄ and 50 mmol/L LiBr in DMF;

Flow rate: 1.0 mL/min

Column temperature: 40° C.

Detector: RI

Calibration curve: Polystyrene was used to prepare a calibration curve.

The high-molecular-weight emulsifier is not particularly limited, and examples thereof include at least one selected from the group consisting of acrylates/C10-30 alkyl acrylate crosspolymer, ammonium acryloyldimethyltaurate/beheneth-25 methacrylate crosspolymer, hydroxyethyl acrylate/sodium acryloyldimethyl taurate copolymer, PEG modified crosspolymer/copolymer siloxane, polyether modified crosspolymer/copolymer siloxane, stearoxy hydroxypropyl methylcellulose, and polyoxyethylene. Among them, acrylates/C10-30 alkyl acrylate crosspolymer is preferred from the viewpoints of emulsion stability, storage stability of the platinum catalyst, and the like.

The blending amount of the high-molecular-weight emulsifier can be 0.01% by mass or more, 0.05% by mass or more, 0.1% by mass or more, or 0.2% by mass or more, and can be 5% by mass or less, 4% by mass or less, 3% by mass or less, 2% by mass or less, or 1% by mass or less with respect to the total amount of the composition from the viewpoints of uniform dispersibility (emulsifiability) of the catalyst in the composition, storage stability of the catalyst, and the like. The blending amount of the high-molecular-weight emulsifier is preferably 2% by mass or less from the viewpoint of film durability and the like.

The weight average molecular weights of common emulsifiers (surfactants) other than the high-molecular-weight emulsifier can be less than 500, 450 or less, or 400 or less, and can be 100 or more, 150 or more, or 200 or more.

The HLBs of common emulsifiers (surfactants) other than the high-molecular-weight emulsifier can be 2.0 or more, 3.0 or more, or 4.0 or more, and can be 10.0 or less, 9.0 or less, or 8.0 or less.

«Coating-type Body Corrective Film Formation Agent»

The oil-in-water composition of the present disclosure described above can be suitably used as a second agent for a coating-type body corrective film formation agent containing a first agent and the second agent. Such a formation agent can form a body corrective film, for example, by applying the first agent to a body surface to form a first agent layer, and then applying the second agent to the first agent layer to allow cross-linking of the first agent layer.

In some embodiments, the application performance of a coating-type body corrective film formation agent can be evaluated in terms of viscosity using a Type B viscometer (Vismetron, manufactured by Shibaura Systems Co., Ltd.). The viscosities of the first agent and the second agent in the coating-type body corrective film formation agent of the present disclosure immediately after the preparation, measured at 25° C., 60 rpm (rotor No. 3 or No. 4), can be, for example, 100 mPa·s or more, 500 mPa·s or more, 1,000 mPa·s or more, 2,000 mPa·s or more, 5,000 mPa·s or more, 7,500 mPa·s or more, 10,000 mPa·s or more, or 15,000 mPa·s or more, and can be, for example, 1,000,000 mPa·s or less, 750,000 mPa·s or less, 500,000 mPa·s or less, 250,000 mPa·s or less, 200,000 mPa·s or less, 175,000 mPa·s or less, 150,000 mPa·s or less, 125,000 mPa·s or less, 100,000 mPa·s or less, or 80,000 mPa·s or less. Among them, from the viewpoints of smooth application performance, prevention of dripping from a skin, and the like, the first agent and the second agent in the coating-type body corrective film formation agent immediately after the preparation preferably have a viscosity of 20,000 mPa·s or less, 15,000 mPa·s or less, or 10,000 mPa·s or less, and preferably have a viscosity of 3,000 mPa·s or more, 5,000 mPa·s or more, or 7,000 mPa·s or more.

In some embodiments, from the viewpoints of smooth application performance, prevention of dripping from a skin, and the like, the viscosities of the first agent and the second agent in the coating-type body corrective film formation agent of the present disclosure 2 weeks after the preparation, measured at 25° C., 60 rpm (rotor No. 3), are preferably 50,000 mPa·s or less, 30,000 mPa·s or less, or 15,000 mPa·s or less, and are preferably 5,000 mPa·s or more, 7,000 mPa·s or more, or 10,000 mPa·s or more.

In some embodiments, the film performance of the body corrective film can be evaluated, for example, based on the presence or absence of tearing of the body corrective film at the time of peeling off from the skin. For example, when tearing of the applied body corrective film occurs at 15% or less, 10% or less, or 5% or less of the total, it can be said that the film performance is excellent. The lower limit of the occurrence of tearing is not particularly restricted, and can be, for example, 0% or more, or more than 0%. In addition, the film performance can be evaluated by tensile strength, elongation at break, or the like as described below.

<First Agent>

The first agent constituting the coating-type body corrective film formation agent of the present disclosure contains at least one selected from the group consisting of the second unsaturated organopolysiloxane and the second hydride functionalized polysiloxane. It is noted that, in cases where the first agent only contains the second unsaturated organopolysiloxane out of the second unsaturated organopolysiloxane and the second hydride functionalized polysiloxane, the second agent composed of the oil-in-water composition described above contains the first hydride functionalized polysiloxane described above, while in cases where the first agent only contains the second hydride functionalized polysiloxane out of the second unsaturated organopolysiloxane and the second hydride functionalized polysiloxane, the second agent contains the first unsaturated organopolysiloxane.

The first agent may be, for example, in an anhydrous form, or in an oil-in-water or water-in-oil form, and from the viewpoint of drying and cross-linking the first agent after application of the first agent to a body surface, it is advantageous for the first agent to be in an anhydrous form. As used herein, the term “water-in-oil” is intended to mean a composition that is in a state where water droplets are dispersed in a dispersion medium containing an oil. Examples of such a composition may include a composition prepared by shaking a liquid that is in a state showing separation into water and an oil, to forcibly disperse water droplets in a dispersion medium containing the oil, and an emulsion composition prepared by blending an emulsifier to disperse water droplets in a dispersion medium containing an oil.

When the agent is in an anhydrous form, the agent usually does not require a preservative against bacteria or mold, and hence can be stored for a longer period of time than emulsions with similar ingredients. Herein, “anhydrous” in the present disclosure is intended to mean that a composition contains no water, or that the water content is as low as 10% by mass or less, 5% by mass or less, 2% by mass or less, 1% by mass or less, or 0.1% by mass or less.

Since the first agent is applied to a body surface by application or the like, the first agent preferably has a glass transition temperature that is not more than body temperature, from the viewpoint of application performance. For example, the glass transition temperature can be 37° C. or lower, 25° C. or lower, 10° C. or lower, or 0° C. or lower. The lower limit of the glass transition temperature is not particularly restricted, and can be, for example, −30° C. or higher, −20° C. or higher, or −10° C. or higher. Here, “glass transition temperature” refers to a temperature at which transition from a solid state to a liquid state occurs, and can be measured, for example, using a differential scanning calorimeter (DSC) in accordance with ASTM D3418-03.

(Second Unsaturated Organopolysiloxane)

As the second unsaturated organopolysiloxane, the same material as the first unsaturated organopolysiloxane described above can be used.

The blending amount of the second unsaturated organopolysiloxane in the first agent may be adjusted as appropriate according to the film performance required or the like, and there is no particular restriction. For example, the blending amount of the second unsaturated organopolysiloxane with respect to the total amount of the first agent can be 5% by mass or more, 10% by mass or more, 20% by mass or more, 30% by mass or more, 35% by mass or more, or 40% by mass or more, and can be 90% by mass or less, 80% by mass or less, 70% by mass or less, 60% by mass or less, 50% by mass or less, or 45% by mass or less.

(Second Hydride Functionalized Polysiloxane)

As the second hydride functionalized polysiloxane, the same material as the first hydride functionalized polysiloxane described above can be used.

The blending amount of the second hydride functionalized polysiloxane in the first agent may be adjusted as appropriate according to the film performance required or the like, and there is no particular restriction. For example, the amount of the second hydride functionalized polysiloxane with respect to the total amount of the first agent can be 1% by mass or more, 3% by mass or more, or 5% by mass or more, and can be 75% by mass or less, 60% by mass or less, 50% by mass or less, 40% by mass or less, 30% by mass or less, 20% by mass or less, or 10% by mass or less.

(Other Polymers)

The first agent can optionally contain polymers other than the second unsaturated organopolysiloxane and the second hydride functionalized polysiloxane. The other polymers can be used singly or in combination of two or more kinds thereof.

In one embodiment, the other polymers can have a viscosity of from 0.7 cSt to 50,000 cSt at 25° C. The lower limit of such a viscosity can be 1 cSt or more, 6 cSt or more, 10 cSt or more, 20 cSt or more, 50 cSt or more, 100 cSt or more, 200 cSt or more, 300 cSt or more, 400 cSt or more, 750 cSt or more, 1,000 cSt or more, 1,500 cSt or more, 2,000 cSt or more, 2,500 cSt or more, 3,000 cSt or more, 3,500 cSt or more, or 4000 cSt or more. The upper limit of the viscosity can be 45,000 cSt or less, 40,000 cSt or less, 35,000 cSt or less, 30,000 cSt or less, 25,000 cSt or less, 20,000 cSt or less, 15,000 cSt or less, 12,000 cSt or less, 10,000 cSt or less, 5,000 cSt or less, 4,000 cSt or less, 2,000 cSt or less, 1,500 cSt or less, or 1,000 cSt or less.

In one embodiment, the other polymers can have an average molecular weight of from 180 Da to 80,000 Da. The lower limit of such an average molecular weight can be 500 Da or more, 800 Da or more, 1,500 Da or more, 3,000 Da or more, 6,000 Da or more, 9,400 Da or more, 10,000 Da or more, 15,000 Da or more, 20,000 Da or more, 30,000 Da or more, 40,000 Da or more, 50,000 Da or more, 55,000 Da or more, 60,000 Da or more, or 62,000 Da or more. The upper limit of the average molecular weight can be 75,000 Da or less, 70,000 Da or less, 65,000 Da or less, or 63,000 Da or less.

Preferred examples of the other polymers include one or more organopolysiloxanes containing at least one alkenyl functional group on average and having a viscosity of from 0.7 to 50,000 cSt at 25° C.

Specific examples of the other polymers that can be used include at least one selected from the group consisting of vinyl-terminated polydimethylsiloxane, vinyl-terminated diphenylsiloxane-dimethylsiloxane copolymer, vinyl-terminated polyphenylmethylsiloxane, vinylphenylmethyl-terminated vinylphenylsiloxane-phenylmethylsiloxane copolymer, vinyl-terminated trifluoropropylmethylsiloxane-dimethylsiloxane copolymer, vinyl-terminated diethylsiloxane-dimethylsiloxane copolymer, vinylmethylsiloxane-dimethylsiloxane copolymer, trimethylsiloxy-terminated vinylmethylsiloxane-dimethylsiloxane copolymer, silanol-terminated vinylmethylsiloxane-dimethylsiloxane copolymer, vinyl-terminated vinyl rubber, vinylmethylsiloxane homopolymer, vinyl T-structure polymer, vinyl Q-structured polymer, an unsaturated organic polymer (such as an unsaturated fatty alcohol, an unsaturated fatty acid, an unsaturated fatty ester, an unsaturated fatty amide, an unsaturated fatty urethane, an unsaturated fatty urea, ceramide, crocetin, lecithin, and sphingosine), monovinyl-terminated poly dimethylsiloxane, vinylmethylsiloxane terpolymer, vinylmethoxysilane homopolymer, vinyl-terminated polyalkylsiloxane polymer, and vinyl-terminated polyalkoxysiloxane polymer. Among them, vinyl-terminated polydimethylsiloxane is preferred, and divinyl dimethicone and 1,3-divinyltetramethyl disiloxane are more preferred.

The blending amount of other polymers in the first agent may be adjusted as appropriate according to the film performance required or the like, and there is no particular restriction. For example, the blending amount of other polymers with respect to the total amount of the first agent can be 0.01% by mass or more, 0.1% by mass or more, 0.3% by mass or more, or 0.5% by mass or more, and can be 20% by mass or less, 15% by mass or less, or 10% by mass or less.

<Ratios of Functional Groups in Second Unsaturated Organopolysiloxane, Second Hydride Functionalized Polysiloxane, and Other Polymers>

In one embodiment, the molar ratio of the Si—H functional groups derived from the second hydride functionalized polysiloxane to the alkenyl functional groups derived from the second unsaturated organopolysiloxane is preferably from 60:1 to 1:5, and more preferably from 45:1 to 15:1.

In one embodiment, the molar ratio of the Si—H functional groups derived from the second hydride functionalized polysiloxane to the alkenyl functional groups derived from other polymers is preferably from 60:1 to 1:5, and more preferably from 45:1 to 15:1.

In one embodiment, the molar ratio of the alkenyl functional groups derived from the second unsaturated organopolysiloxane to the alkenyl functional groups derived from the other polymers is preferably from 100:1 to 1:100, and more preferably from 10:1 to 1:10.

<Second Agent>

In the coating-type body corrective film formation agent of the present disclosure, the oil-in-water composition of the present disclosure described above is used as the second agent.

The above-described other polymers that can be blended in the first agent can be similarly blended in the second agent.

When the first unsaturated organopolysiloxane and the other polymers are used in the second agent, the functional-group ratio between these that may be employed is the same as the functional-group ratio between the second unsaturated organopolysiloxane and the other polymers.

<Optional Ingredients>

The coating-type body corrective film formation agent of the present disclosure may contain various ingredients in the first agent and/or the second agent as appropriate as long as the effects of the present disclosure are not affected.

The optional ingredients are not particularly limited, and examples thereof include a feel modifier, a pressure sensitive adhesion modifier, a spreading promoter, a diluent, an adhesion modifier, an emulsifier (surfactant), an emollient, a solvent, a film-forming agent, a humectant, a preservative, a fiber, a pigment, a dye, an ingredient for thickening the aqueous phase or oil phase (thickener), a protective colloid, a filler, a skin penetration enhancer, an optical modifier, a scattering agent, an adsorbent, a magnetic material, a gaseous transport modifier, a liquid transport modifier, a pH modifier, a sensitizing modifier, and an aesthetic modifier. Such optional ingredients can be used singly or in combination of two or more kinds thereof.

Examples of the filler can include at least one selected from carbon, silver, mica, zinc sulfide, zinc oxide, titanium dioxide, aluminum oxide, clay, chalk, talc, calcite (such as CaCO₃), barium sulfate, zirconium dioxide, polymer beads, silica (such as fumed silica, silica acid, or anhydrous silica), silica aluminate, and calcium silicate, which may be surface-treated. Such a filler can improve physical properties (such as strength) of the body corrective film, and can also serve as a viscosity modifier. Among the fillers, a surface-treated silica, such as a silica treated with a surface treatment agent such as hexamethyldisilazane, polydimethylsiloxane, hexadecylsilane, or methacrylsilane, is preferred. Fumed silica is also preferred, and for example, fumed silica surface-treated with hexamethyldisilazane or the like can also suitably be used.

In one embodiment, the filler can have a specific surface area of from 50 to 500 m²/g. The specific surface area of the filler is preferably from 100 to 350 m²/g, and more preferably from 135 to 250 m²/g. The specific surface area of the filler can be calculated using the BET method.

In one embodiment, the filler can have an area equivalent circle diameter of from 1 nm to 20 μm. The area equivalent circle diameter of the filler is preferably from 2 nm to 1 μm, and more preferably from 5 nm to 50 nm. Here, the area equivalent circle diameter of the filler can be intended, for example, to be in terms of the particle diameter of a circular particle having the same area as projected area of a filler particle observed with a transmission electron microscope. Such an area equivalent circle diameter can be defined as an average value of 10 or more particles.

The blending amount of the filler with respect to the total amount of the first agent or the second agent can be, for example, 1% by mass or more, 3% by mass or more, or 5% by mass or more, and can be 25% by mass or less, 15% by mass or less, or 10% by mass or less.

From the viewpoint of reinforcing properties and the like of the body corrective film, the mass ratio of the total amount of the first and second unsaturated organopolysiloxanes, the first and second hydride functionalized polysiloxanes, and the other polymers to the filler can be from 100:1 to 1:1, and preferably from 50:1 to 2:1, more preferably from 15:1 to 3:1, still more preferably from 10:1 to 4:1, and particularly preferably from 5:1 to 9:1.

At least one selected from the pigment, the dye, and the filler among the optional ingredients is preferably blended in the first agent. In particular, when the pigment or dye is blended into the second agent, and this second agent is applied to the surface to which the first agent is applied, the second agent may harden during the application, and the pigment or dye may easily become localized, which may cause color unevenness. From the viewpoint of preventing the color unevenness, it is advantageous for the pigment or dye to be blended in the first agent. The pigment, the dye, and the filler may be blended in the second agent to an extent that color unevenness does not occur, but it is advantageous for them not to be contained in a second agent.

In one embodiment, in the composition of the present disclosure, one or more agents can be further blended for the first agent and/or the second agent. Examples of such agents include a cosmetic agent, a therapeutic agent, a stimulant-responsive agent, and a drug delivery agent.

Preferred examples of the cosmetic agent include a moisturizing agent, a UV absorber, a skin protectant, a skin calming agent, a skin whitener, a skin brightener, a skin softener, a skin smoothing agent, a skin bleaching agent, a skin exfoliator, a skin tightener, a beauty treatment agent, a vitamin, an antioxidant, a cell signaling agent, a cell modulator, a cell interaction agent, a skin tanning agent, an anti-aging agent, an anti-wrinkle agent, a spot reducer, an α-hydroxy acid, a β-hydroxy acid, and a ceramide.

Preferred examples of the suitable therapeutic agent include a pain reliever, an analgesic, an antipruritic agent, an anti-acne agent (such as beta-hydroxy acid, salicylic acid, or benzoyl peroxide), an anti-inflammatory agent, an antihistamine, a corticosteroid, a NSAID (nonsteroidal anti-inflammatory drug), an antiseptic, an antibiotic, an antibacterial agent, an antifungal agent, an antiviral agent, an antiallergic agent, an anti-irritant agent, an insect repellent, a phototherapeutic agent, a blood coagulant, an anti-neoplastic agent, an immune system stimulant, an immune system suppressant, a coal tar, anthralin, fluocinonide, methotrexate, cyclosporine, pimecrolimus, tacrolimus, azathioprine, fluorouracil, a ceramide, a counterirritant, and a skin-cooling compound.

Preferred examples of the agent include an antioxidant, a vitamin, a vitamin D₃ analog, a retinoid, a mineral, a mineral oil, vaseline, a fatty acid, a plant extract, a polypeptide, an antibody, a protein, a sugar, a humectant, and an emollient.

«Method for Using Coating-type Body Corrective Film Formation Agent»

The coating-type body corrective film formation agent of the present disclosure can be used, for example, for cosmetic or medical purposes. Herein, the method for using the coating-type body corrective film formation agent of the present disclosure does not encompass a method of operating, treating, or diagnosing a human.

The method for using a coating-type body corrective film formation agent of the present disclosure is not particularly limited, and examples thereof include: a method in which after the first agent is applied to the surface of a body to form a first agent layer, the second agent is applied on the first agent layer to allow cross-linking, to form a body corrective film; a method in which after the second agent is applied to the surface of a body to form a second agent layer, the first agent is applied on the second agent layer to allow cross-linking, to form a body corrective film; and a method in which after the first agent and the second agent are mixed to prepare a mixture, the mixture is applied to the surface of a body to allow cross-linking, to form a body corrective film. From the viewpoint of obtaining a uniform body corrective film having little unevenness, the method of use is preferably a method in which the first agent is applied to the body surface to form a first agent layer, and then the second agent is applied on this first agent layer to allow cross-linking, to form a body corrective film. Here, for the first agent and the second agent, the above-described materials and the like can be similarly used.

This method may be performed only once, or may be performed a plurality of times on the formed body corrective film.

In some embodiments, before the first agent, the second agent, or the mixture containing the first agent and the second agent is applied to the body surface, a cosmetic may be applied to the body surface; after the first agent is applied to the body surface to form the first agent layer, and a cosmetic is applied on the first agent layer, the second agent may be applied to cover the cosmetic; after the second agent is applied to the body surface to form the second agent layer, and a cosmetic is applied on the second agent layer, the first agent may be applied to cover the cosmetic; or after the body corrective film is formed, a cosmetic may be applied to the film.

The cosmetic is not particularly limited. For example, a skin care cosmetic such as a beauty serum, a skin lotion, or a milky lotion; a sunscreen cosmetic (sun block cosmetic); a base cosmetic; or a make-up cosmetic such as a foundation, a gloss, a lipstick, an eye shadow, or a manicure; or a cosmetic having a combination of two or more functions of these cosmetics; can be used.

In some embodiments, a method for using a coating-type body corrective film formation agent of the present disclosure may also be utilized as a cosmetic method. The term “cosmetic method” means application of the coating-type body corrective film formation agent of the present disclosure to the body surface to form a body corrective film to beautify conditions of the body surface, or a method thereof, which is different from a method of operating, treating, or diagnosing a human.

The method of applying the first agent or the second agent to the body surface or the cosmetic-applied layer, or to the first agent layer or the second agent layer, is not particularly restricted, and for example, a means of spreading it with a finger or the like, spray application, transfer, or the like can be employed for the application.

When the first agent and the second agent show separation into water and oil, it is preferred to shake these agents to force them to be a two-phase system (oil-in-water or water-in-oil) from the viewpoints of shortening the cross-linking reaction time, the film durability, and the like.

<Application Site>

The coating-type body corrective film formation agent of the present disclosure can be applied on the surface of a skin in any part of a body, namely, on any part of the body surface. For example, the agent can be applied to the skin surface of a face (a lip, an eye, a nose, a cheek, a forehead, or the like), a neck, an ear, a hand, an arm, a leg, a foot, a chest, a belly, a back, or the like, as appropriate. Here, the skin also encompasses, for example, a nail, which is formed as a result of hardening due to a change in keratin of the epidermis of a skin.

«Kit Containing Coating-Type Body Corrective Film Formation Agent»

The coating-type body corrective film formation agent of the present disclosure may be provided as a kit containing a first agent and a second agent constituting such a formation agent. In addition to the first agent and the second agent, the kit may also contain an optional member, such as a member to facilitate application of the first agent or the like to a body surface, or various cosmetics described above.

Examples of such an optional member include a usage instruction, a brush, a cotton swab, a cutter, scissors, various cosmetics described above, a cleanser for removing the body corrective film from a body surface, and a mirror. Here, “usage instruction” can encompass, in addition to a general usage instruction attached to the kit in the form of a document, for example, an instruction printed on a packaging container that contains the kit or on a packaging container such as a tube that injects the first agent or the like.

In one embodiment, to prevent contact between the first agent and the second agent, for example, these agents may be contained in separate containers, or may be contained in separate compartments of a container having two or more compartments, in the kit. These agents contained may be configured such that they are applied one at a time, or mixed together before or at the time of use.

«Body Corrective Film»

<Thickness>

The thickness of the body corrective film prepared using the coating-type body corrective film formation agent of the present disclosure described above is not particularly limited, and can be adjusted as appropriate, taking into consideration, for example, breathability, invisibility, compressibility, and closure to a skin. The thickness of the body corrective film can be, for example, 0.5 μm or more, 1 μm or more, 10 μm or more, 30 μm or more, or 40 μm or more. The upper limit of the thickness is not particularly limited, and can be, for example, 150 μm or less, 100 μm or less, 90 μm or less, 80 μm or less, 70 μm or less, 60 μm or less, or 50 μm or less. Here, the thickness can be defined as an average value calculated by measuring the thickness of any portion of the body corrective film five times using a High-Accuracy Digimatic Micrometer (MDH-25 MB, manufactured by Mitutoyo Corporation).

<Performances>

A body corrective film prepared from the coating-type body corrective film formation agent of the present disclosure can provide excellent results on a variety of performances such as those shown below.

(Adhesive Strength)

In some embodiments, the obtained body corrective film can exhibit favorable adhesive strength to a body surface. Such adhesive strength can be evaluated alternatively as the adhesive strength of the body corrective film applied on a polypropylene substrate. The adhesive strength of the body corrective film on the polypropylene substrate can achieve 2 N/m or more, 5 N/m or more, 8 N/m or more, 10 N/m or more, or 15 N/m or more. The upper limit of such an adhesive strength is not particularly limited, and can be, for example, from the viewpoint of peelability from a skin or the like, 200 N/m or less, 100 N/m or less, 80 N/m or less, 50 N/m or less, or 30 N/m or less. Here, the adhesive strength can be measured using an Instron device in accordance with the ASTM C794 peel-off adhesion test.

(Tensile Strength)

In some embodiments, the obtained body corrective film can exhibit favorable tensile strength. The tensile strength of the body corrective film can achieve 0.05 MPa or more, 0.10 MPa or more, 0.20 MPa or more, or 0.50 MPa or more. The upper limit of the tensile strength is not particularly restricted, and can be, for example, 5.0 MPa or less, 3.0 MPa or less, 2.0 MPa or less, or 1.0 MPa or less. Here, the tensile strength can be measured using an Instron device in accordance with the ASTM D5083 elongation tensile test.

(Elongation at Break)

In some embodiments, the obtained body corrective film can exhibit favorable elongation at break. The elongation at break of the body corrective film can achieve 25% or more, 50% or more, 100% or more, 200% or more, or 400% or more. The upper limit of the elongation at break is not particularly restricted, and can be, for example, 1,500% or less, 1,200% or less, 1,000% or less, 800% or less, or 600% or less. Here, the elongation at break can be measured using an Instron device in accordance with the ASTM D5083 elongation tensile test.

(Oxygen Transmission Rate)

In some embodiments, the obtained body corrective film can exhibit a favorable oxygen transmission rate. In a body corrective film having a thickness of 300 μm, the oxygen transmission rate of the body corrective film can achieve 5×10⁻⁹ cm³/(cm²·s) or more, 5×10⁻⁷ cm³/(cm²·s) or more, or 5×10⁻⁵ cm³/(cm²·s) or more. The upper limit of the oxygen transmission rate is not particularly restricted, and can be, for example, 5 cm³/(cm²·s) or less, 0.5 cm³/(cm²·s) or less, 5×10⁻² cm³/(cm²·s) or less, 5×10⁻³ cm³/(cm²·s) or less, or 5×10⁻⁴ cm³/(cm²·s) or less. Here, the oxygen transmission rate can be measured using a MOCON device in accordance with the ASTM F2622 test method for oxygen gas transmission rate through plastic film and sheeting.

(Water Vapor Transmission Rate)

In some embodiments, the obtained body corrective film can exhibit a favorable water vapor transmission rate. In a body corrective film having a thickness of 300 μm, the water vapor transmission rate of the body corrective film can achieve 1×10⁻⁹ cm³/(cm²·s) or more, 1×10⁻⁸ cm³/(cm²·s) or more, or 1×10⁻⁷ cm³/(cm²·s) or more. The upper limit of the water vapor transmission rate is not particularly restricted, and can be, for example, 1.5×10⁻¹ cm³/(cm²·s) or less, 1.5×10⁻² cm³/(cm²·s) or less, 1×10⁻⁴ cm³/(cm²·s) or less, 1×10⁻⁵ cm³/(cm²·s) or less, or 1×10⁻⁶ cm³/(cm²·s) or less. Here, the water vapor transmission rate can be measured using a MOCON device in accordance with the ASTM F1249 test method for water vapor transmission rate through plastic film and sheeting.

### EXAMPLES

The present invention will be described in more detail by way of Examples below, but is not limited thereto. Unless otherwise noted, the blending amounts are shown below in % by mass.

«Evaluation Test»

The tests shown below were performed using test samples obtained by the production method described below. The results are summarized in Tables 1 to 5.

<Test for Evaluating Cross-Linking Properties of Film: Shortening of Cross-Linking Reaction Time>

In an environment at 25° C.±1° C. and a relative humidity of 50%±2%, about 0.1 g of a first agent was spread on the skin to form a substantially circular first agent layer having a diameter of about 4 cm, and then about 0.1 g of a second agent was applied with a finger to the first agent layer, followed by spreading the second agent around with a finger in a circular motion. The cross-linking reaction time of each film was defined as from the point when the second agent came into contact with the first agent layer to the point when curing of the surface of the film had proceeded to a level at which no damage occurred even by touching with a finger. The cross-linking property of the film was evaluated according to the following evaluation criteria. In the following evaluation criteria, ratings of A to C can be considered acceptable, and a rating of D can be considered unacceptable:
A: The cross-linking reaction time was within 40 seconds;
B: The cross-linking reaction time was more than 40 seconds to 1 minute or less;
C: The cross-linking reaction time was more than 1 minute to 2 minutes or less; and
D: The cross-linking reaction time was more than 2 minutes.

<Visual Evaluation Test for Film Appearance: Durability of Film on Skin>

In an environment at 25° C.±1° C. and a relative humidity of 50%±2%, about 0.1 g of a first agent was spread on a forearm to form a substantially circular first agent layer having a diameter of about 4 cm, and then about 0.1 g of a second agent was applied with a finger to the first agent layer, followed by spreading the second agent around with a finger in a circular motion to prepare a body corrective film. The obtained film was visually observed for the presence of defects such as peeling or damage to evaluate the durability of the film on a skin according to the evaluation criteria described below. In the following evaluation criteria, ratings of A and B can be considered acceptable, and a rating of C can be considered unacceptable:
A: No peeling of or damage to the film was observed;
B: Slight peeling of or damage to the film occurred;
C: Clear peeling of or damage to the film occurred.

<Abrasion Resistance Evaluation Test: Film Durability>

About 0.1 g of a first agent was spread on the surface of a 3 cm×10 cm artificial skin (SUPPRARE™: Idemitsu Technofine Co., Ltd.) to form a substantially circular first agent layer having a diameter of about 4 cm, and then about 0.1 g of a second agent was applied with a finger to the first agent layer, followed by spreading the second agent around with a finger in a circular motion to prepare a body corrective film. The artificial skin to which the film was applied was then set on a Crock meter (Hanchen), and the film was repeatedly rubbed with a load of 1 N applied to the film while the number of repetition was counted until damage to the film occurred. In the following evaluation criteria, ratings of A to C can be considered acceptable, and a rating of D can be considered unacceptable. The number of repetition until the occurrence of damage is indicated in parentheses in the table.
A: The number of repetition until the occurrence of damage in the film was 20 times or more;
B: The number of repetition until the occurrence of damage in the film was from 10 times to 19 times;
C: The number of repetition until the occurrence of damage in the film was from 5 times to 9 times; and
D: The number of repetition until the occurrence of damage in the film was 4 times or less.

<Emulsifiability Evaluation Test: Uniformity of Emulsion Particles>

In an environment at 25° C.±1° C., the emulsion particle (oil droplet) sizes immediately after the preparation of an oil-in-water composition or a water-in-oil composition were measured with a light microscope (BX53, manufactured by OLYMPUS), and the emulsifiability was evaluated according to the evaluation criteria described below. In the emulsification evaluation, 10 emulsion particles were arbitrarily selected, and the largest and smallest of these emulsion particles were excluded. For the remaining 8 emulsion particles, the sizes of the largest and smallest emulsion particles were compared to determine the emulsion particle size variation. In the following evaluation criteria, ratings of A and B can be considered acceptable, and a rating of C can be considered unacceptable:
A: The emulsion particle size variation was within 5 μm;
B: The emulsion particle size variation was more than 5 μm and 10 μm or less; and
C: The emulsion particle size variation was more than 10 μm.

### Test Example 1: Determination of Cross-Linking Properties, Durability, and the Like of Films with Different Formulations of Second Agents

In Test Example 1, the cross-linking properties, durability, and the like of films with different formulations of second agents in coating-type body corrective film formation agents were investigated. The results are shown in Tables 1 and 2.

<Method of Producing First Agent Used with Oil-Single-Phase or Water-In-Oil Second Agent>

A first agent was prepared by uniformly mixing 45 parts by mass of divinyl dimethicone at 165,000 cSt as a second unsaturated organopolysiloxane, 10 parts by mass of hydrogen dimethicone at 45 cSt as a second hydride functionalized polysiloxane, 7.5 parts by mass of silica silylate as a filler, and 37.5 parts by mass of a mixture of dimethicone and trisiloxane as an oil.

<Method of Producing First Agent Used with Oil-In-Water Second Agent>

A first agent was prepared by uniformly mixing 30 parts by mass of divinyl dimethicone at 165,000 cSt as a second unsaturated organopolysiloxane, 7 parts by mass of hydrogen dimethicone at 45 cSt as a second hydride functionalized polysiloxane, 6 parts by mass of silica silylate as a filler, and 57 parts by mass of a mixture of dimethicone and trisiloxane as an oil.

<Method of Producing Oil-single-phase Second Agents>

Using the Formulations Shown in Table 1, Oil-Single-Phase Second Agents were Produced by the following method. Here, the numbers shown below correspond to the numbers representing the ingredients on the left side of the formulations in Table 1.

### Comparative Example 1

The materials of Nos. 6 and 7 were added to a mixture of the oils of Nos. 1, 3, and 4, and uniformly mixed to prepare an oil-single-phase second agent of Comparative Example 1.

<Method of Producing Second Agents as Water-In-Oil Compositions>

Using the formulations shown in Table 1, second agents as water-in-oil compositions were produced by the following method. Here, the numbers shown below correspond to the numbers representing the ingredients on the left side of the formulations in Table 1.

### Comparative Example 2

The materials of Nos. 1 to 4, 6, and 7 were uniformly mixed to prepare an oil-phase part.

The materials of Nos. 8 to 12 were uniformly mixed to prepare an aqueous-phase part.

The aqueous-phase part was added to the oil-phase part, and uniformly mixed to prepare a second agent as a water-in-oil composition of Comparative Example 2.

### Comparative Examples 3 to 7

Second agents as water-in-oil compositions of Comparative Examples 3 to 7 were prepared in the same manner as in Comparative Example 2 except that their formulations in Table 1 were used.

<Method of Producing Second Agents as Oil-In-Water Compositions>

Using the formulations shown in Table 2, second agents as oil-in-water compositions were produced by the following method. Here, the numbers shown below correspond to the numbers representing the ingredients on the left side of the formulations in Table 2.

### Example 1

The materials of Nos. 1 to 4 and 7 to 9 were uniformly mixed to prepare an aqueous-phase part, and the catalyst of No. 5 was then added thereto, followed by uniformly mixing the resulting mixture to prepare a second agent as an oil-in-water composition of Example 1.

### Examples 2 to 7

Second agents as oil-in-water compositions of Examples 2 to 7 were prepared in the same manner as in Example 1 except that their formulations in Table 2 were used. The emulsifier of No. 6 was blended when the aqueous-phase part was prepared.

TABLE 1

| | | | | Water-in-oil | | | | | |
|---|---|---|---|---|---|---|---|---|---|
| | Formulation of second agent | | Oil single phase | Com. | Com. | Com. | Com. | Com. | Com. |
| No. | Ingredient | Ingredient name | Com. Ex. 1 | Ex. 2 | Ex. 3 | Ex. 4 | Ex. 5 | Ex. 6 | Ex. 7 |
| 1 | Oil | Dimethicone and | 30.0 | 10.0 | 10.0 | 10.0 | 10.0 | 10.0 | 20.0 |
| | | dimethicone | | | | | | | |
| | | crosspolymer | | | | | | | |
| 2 | Emulsifier | Mixture¹⁾ of | — | 1.0 | 3.0 | 5.0 | 5.0 | 5.0 | 5.0 |
| | (oil) | dimethicone, | | | | | | | |
| | | (dimethicone/(PEG- | | | | | | | |
| | | 10/15)) | | | | | | | |
| | | crosspolymer, | | | | | | | |
| | | dipropylene glycol, | | | | | | | |
| | | and tocopherol | | | | | | | |
| 3 | Oil | Dimethicone | 10.0 | 10.0 | 10.0 | 10.0 | 10.0 | 10.0 | 10.0 |
| | | (viscosity: 6 cSt) | | | | | | | |
| 4 | | Dimethicone | 55.5 | 15.0 | 13.0 | 11.0 | 15.0 | 15.0 | 15.0 |
| | | (viscosity: 1.5 cSt) | | | | | | | |
| 5 | | Coconut alkane and | — | — | — | — | 10.0 | 20.0 | 20.0 |
| | | coco- | | | | | | | |
| | | caprylate/caprate | | | | | | | |
| 6 | Filler | Silica | 3.0 | 3.0 | 3.0 | 3.0 | 3.0 | 3.0 | 3.0 |
| 7 | Catalyst | Mixture²⁾ of vinyl | 1.5 | 1.5 | 1.5 | 1.5 | 1.5 | 1.5 | 1.5 |
| | | dimethicone, divinyl | | | | | | | |
| | | disiloxane, and | | | | | | | |
| | | platinum catalyst | | | | | | | |
| 8 | Salt | Sodium chloride | — | 0.5 | 0.5 | 0.5 | 0.5 | 0.5 | 0.5 |
| 9 | Preservative | Phenoxyethanol | — | 0.5 | 0.5 | 0.5 | 0.5 | 0.5 | 0.5 |
| 10 | Polyhydric | Dipropylene glycol | — | 5.0 | 5.0 | 5.0 | 5.0 | 5.0 | 5.0 |
| | alcohol | | | | | | | | |
| 11 | Moisturizing | Glycerol | — | 4.0 | 4.0 | 4.0 | 4.0 | 4.0 | 4.0 |
| | agent | | | | | | | | |
| 12 | Water | Ion exchanged water | — | Balance | Balance | Balance | Balance | Balance | Balance |

| Total | | 100 | 100 | 100 | 100 | 100 | 100 | 100 |
|---|---|---|---|---|---|---|---|---|
| Blending | (i) Oil content (part by mass) | 95.5 | 36.0 | 36.0 | 36.0 | 50.0 | 60.0 | 70.0 |
| ratio of | excluding reactive polymer (vinyl | | | | | | | |
| catalyst | dimethicone) | | | | | | | |
| | (ii) Platinum catalyst content (part | 0.030 | 0.030 | 0.030 | 0.030 | 0.030 | 0.030 | 0.030 |
| | by mass) | | | | | | | |
| | Blending ratio (%) of catalyst to | 0.031 | 0.083 | 0.083 | 0.083 | 0.060 | 0.050 | 0.043 |
| | oil: {(ii) × 100}/(i) | | | | | | | |
| Evaluation | Cross-linking properties of film | D | D | D | D | D | D | D |
| | Appearance of film | C | C | C | C | C | C | C |
| | Abrasion resistance (number of | D(1) | D(4) | D(3) | D(2) | D(2) | D(2) | D(1) |
| | repetition) | | | | | | | |
| | Emulsifiability | N.D. | B | A | A | A | A | A |

¹⁾Since this is a mixture of (dimethicone/(PEG-10/15)) crosspolymer that function as emulsifiers and oils, it is considered as an oil in the calculation of the blending ratio of catalysts to oils.
²⁾Divinyl disiloxane in the mixture is considered as an oil in the calculation of the blending ratio of catalysts to oils. The ratios of the ingredients in the mixture: 2% by mass of platinum, 3% by mass of divinyl disiloxane, and 95% by mass of vinyl dimethicone.

TABLE 2

| | Formulation of second agent | | Oil-in-water | | | | | | |
|---|---|---|---|---|---|---|---|---|---|
| No. | Ingredient | Ingredient name | Ex. 1 | Ex. 2 | Ex. 3 | Ex. 4 | Ex. 5 | Ex. 6 | Ex. 7 |
| 1 | Water | Ion exchanged | Balance | Balance | Balance | Balance | Balance | Balance | Balance |
| | | water | | | | | | | |
| 2 | Moisturizing | Glycerol | 4.0 | 4.0 | 4.0 | 4.0 | 4.0 | 4.0 | 4.0 |
| | agent | | | | | | | | |
| 3 | Polyhydric | Dipropylene | 5.0 | 5.0 | 5.0 | 5.0 | 5.0 | 5.0 | 5.0 |
| | alcohol | glycol | | | | | | | |
| 4 | Thickener | Carboxyvinyl | 0.3 | 0.3 | 0.3 | 0.3 | 0.3 | 0.3 | 0.3 |
| | | polymer | | | | | | | |
| 5 | Catalyst | Mixture¹⁾ of | 1.5 | 1.5 | 1.5 | 1.5 | 1.5 | 1.5 | 1.5 |
| | | vinyl | | | | | | | |
| | | dimethicone, | | | | | | | |
| | | divinyl | | | | | | | |
| | | disiloxane, and | | | | | | | |
| | | platinum catalyst | | | | | | | |
| 6 | Emulsifier | POE(60) | — | 0.1 | 0.1 | 0.1 | 1.0 | 3.0 | 5.0 |
| | | hydrogenated | | | | | | | |
| | | castor oil | | | | | | | |
| 7 | pH adjuster | Potassium | q.s. | q.s. | q.s. | q.s. | q.s. | q.s. | q.s. |
| | | hydroxide | | | | | | | |
| 8 | Lower alcohol | Denatured | 20.0 | 20.0 | 20.0 | 20.0 | 20.0 | 20.0 | 20.0 |
| | | alcohol | | | | | | | |
| 9 | Preservative | Phenoxyethanol | 0.5 | 0.5 | 0.5 | 0.5 | 0.5 | 0.5 | 0.5 |

| Total | | 100 | 100 | 100 | 100 | 100 | 100 | 100 |
|---|---|---|---|---|---|---|---|---|
| Blending | (i) Oil content (part by mass) excluding | 0.045 | 0.045 | 0.045 | 0.045 | 0.045 | 0.045 | 0.045 |
| ratio of | reactive polymer (vinyl dimethicone) | | | | | | | |
| catalyst | (ii) Platinum catalyst content (part by | 0.030 | 0.030 | 0.030 | 0.030 | 0.030 | 0.030 | 0.030 |
| | mass) | | | | | | | |
| | Blending ratio (%) of catalyst to oil: | 67 | 67 | 67 | 67 | 67 | 67 | 67 |
| | {(ii) × 100}/(i) | | | | | | | |
| Evaluation | Cross-linking properties of film | A | A | A | A | A | B | B |
| | Appearance of film | A | A | A | A | A | A | B |
| | Abrasion resistance (number of repetition) | A(40) | B(14) | B(14) | B(13) | B(14) | B(12) | C(8) |
| | Emulsifiability | C | A | A | A | A | A | A |

¹⁾Divinyl disiloxane in the mixture is considered as an oil in the calculation of the blending ratio of catalysts to oils. The ratios of the ingredients in the mixture: 2% by mass of platinum, 3% by mass of divinyl disiloxane, and 95% by mass of vinyl dimethicone.

<Results>

As can be seen from the results in Tables 1 and 2, each oil-in-water formulation as the second agent can shorten the cross-linking reaction time of the film and also improve the durability of the film.

Comparison between Examples 1 and 2 shows that use of an emulsifier can improve the emulsifiability. Comparison among Examples 5 to 7 shows that the blending amount of the emulsifier is preferably 5.0% by mass or less in view of film durability.

### Test Example 2: Determination of Cross-Linking Properties, Durability, and the Like of Films with Different Emulsifiers in Second Agents

In Test Example 2, the cross-linking properties, durability, and the like of films with different emulsifiers in second agents in coating-type body corrective film formation agents were investigated. The results are shown in Table 3.

<Method of Producing First Agent Used with Second Agent>

A first agent was prepared by uniformly mixing 20 parts by mass of divinyl dimethicone at 165,000 cSt as a second unsaturated organopolysiloxane, 10 parts by mass of hydrogen dimethicone at 45 cSt as a second hydride functionalized polysiloxane, 40 parts by mass of ion exchanged water, 1 part by mass of sodium chloride, 1 part by mass of 1,3-butylene glycol, 0.5 parts by mass of phenoxyethanol, and 27.5 parts by mass of a mixture of dimethicone and trisiloxane as an oil.

<Method of Producing Second Agents as Oil-In-Water Compositions>

Using the formulations shown in Table 3, second agents as oil-in-water compositions were produced by the following method. Here, the numbers shown below correspond to the numbers representing the ingredients on the left side of the formulations in Table 3.

### Example 8

The materials of Nos. 1 to 4 and 18 to 21 were uniformly mixed to prepare an aqueous-phase part, and the catalyst of No. 17 was then added thereto, followed by uniformly mixing the resulting mixture to prepare a second agent as an oil-in-water composition of Example 8.

### Examples 9 to 20

Second agents as oil-in-water compositions of Examples 9 to 20 were prepared in the same manner as in Example 1 except that their formulations in Table 3 were used.

TABLE 3

| | Formulation of second agent | | Oil-in-water | | | | | | |
|---|---|---|---|---|---|---|---|---|---|
| No. | Ingredient | Ingredient name | Ex. 8 | Ex. 9 | Ex. 10 | Ex. 11 | Ex. 12 | Ex. 13 | Ex. 14 |
| 1 | Water | Ion exchanged | Balance | Balance | Balance | Balance | Balance | Balance | Balance |
| | | water | | | | | | | |
| 2 | Moisturizing agent | Glycerol | 20.0 | 20.0 | 20.0 | 20.0 | 20.0 | 20.0 | 20.0 |
| 3 | Thickener | Carboxyvinyl | 0.2 | 0.2 | 0.2 | 0.2 | 0.2 | 0.2 | 0.2 |
| | | polymer | | | | | | | |
| 4 | Emulsifier | PEG-12 | 1.0 | — | — | — | — | — | — |
| | | dimethicone and | | | | | | | |
| | | tocopherol | | | | | | | |
| 5 | | PEG-11 methyl | — | 1.0 | — | — | — | — | — |
| | | ether dimethicone | | | | | | | |
| 6 | | PEG-9 | — | — | 1.0 | — | — | — | — |
| | | dimethicone | | | | | | | |
| 7 | | PEG-100 | — | — | — | 1.0 | — | — | — |
| | | hydrogenated | | | | | | | |
| | | castor oil | | | | | | | |
| 8 | | Beheneth-20 and | — | — | — | — | 1.0 | — | — |
| | | tocopherol | | | | | | | |
| 9 | | Steareth-20 | — | — | — | — | — | 1.0 | — |
| 10 | | PEG-100 stearate | — | — | — | — | — | — | 1.0 |
| 11 | | PEG-60 glyceryl | — | — | — | — | — | — | — |
| | | isostearate and | | | | | | | |
| | | tocopherol | | | | | | | |
| 12 | | Polysorbate 60 | — | — | — | — | — | — | — |
| 13 | | Glyceryl stearate | — | — | — | — | — | — | — |
| 14 | | Sorbitan | — | — | — | — | — | — | — |
| | | isostearate | | | | | | | |
| 15 | | Sucrose stearate | — | — | — | — | — | — | — |
| | | and sucrose | | | | | | | |
| | | palmitate | | | | | | | |
| 16 | | Polyglyceryl-10 | — | — | — | — | — | — | — |
| | | laurate | | | | | | | |
| 17 | Catalyst | Mixture¹⁾ of | 1.5 | 1.5 | 1.5 | 1.5 | 1.5 | 1.5 | 1.5 |
| | | vinyl | | | | | | | |
| | | dimethicone, | | | | | | | |
| | | divinyl | | | | | | | |
| | | disiloxane, and | | | | | | | |
| | | platinum catalyst | | | | | | | |
| 18 | pH adjuster | Potassium | q.s. | q.s. | q.s. | q.s. | q.s. | q.s. | q.s. |
| | | hydroxide | | | | | | | |
| 19 | Lower alcohol | Denatured | 10.0 | 10.0 | 10.0 | 10.0 | 10.0 | 10.0 | 10.0 |
| | | alcohol | | | | | | | |
| 20 | Preservative | Phenoxyethanol | 0.5 | 0.5 | 0.5 | 0.5 | 0.5 | 0.5 | 0.5 |
| 21 | Filler | Nylon-12 | 8.0 | 8.0 | 8.0 | 8.0 | 8.0 | 8.0 | 8.0 |
| | | particles, average | | | | | | | |
| | | particle size: 5 μm | | | | | | | |

| Total | | 100 | 100 | 100 | 100 | 100 | 100 | 100 |
|---|---|---|---|---|---|---|---|---|
| Blending | (i) Oil content (part by mass) excluding | 0.045 | 0.045 | 0.045 | 0.045 | 0.045 | 0.045 | 0.045 |
| ratio of | reactive polymer (vinyl dimethicone) | | | | | | | |
| catalyst | (ii) Platinum catalyst content (part by | 0.030 | 0.030 | 0.030 | 0.030 | 0.030 | 0.030 | 0.030 |
| | mass) | | | | | | | |
| | Blending ratio (%) of catalyst to oil: | 67 | 67 | 67 | 67 | 67 | 67 | 67 |
| | {(ii) × 100}/(i) | | | | | | | |
| Evaluation | Cross-linking properties of film | A | A | A | A | A | A | A |
| | Appearance of film | A | A | A | A | A | A | A |
| | Abrasion resistance (number of repetition) | A(50) | B(10) | B(11) | B(13) | B(11) | B(12) | B(10) |
| | Emulsifiability | A | A | A | A | A | A | A |

| | Formulation of second agent | | Oil-in-water | | | | | |
|---|---|---|---|---|---|---|---|---|
| No. | Ingredient | Ingredient name | Ex. 15 | Ex. 16 | Ex. 17 | Ex. 18 | Ex. 19 | Ex. 20 |
| 1 | Water | Ion exchanged | Balance | Balance | Balance | Balance | Balance | Balance |
| | | water | | | | | | |
| 2 | Moisturizing agent | Glycerol | 20.0 | 20.0 | 20.0 | 20.0 | 20.0 | 20.0 |
| 3 | Thickener | Carboxyvinyl | 0.2 | 0.2 | 0.2 | 0.2 | 0.2 | 0.2 |
| | | polymer | | | | | | |
| 4 | Emulsifier | PEG-12 | — | — | — | — | — | — |
| | | dimethicone and | | | | | | |
| | | tocopherol | | | | | | |
| 5 | | PEG-11 methyl | — | — | — | — | — | — |
| | | ether dimethicone | | | | | | |
| 6 | | PEG-9 | — | — | — | — | — | — |
| | | dimethicone | | | | | | |
| 7 | | PEG-100 | — | — | — | — | — | — |
| | | hydrogenated | | | | | | |
| | | castor oil | | | | | | |
| 8 | | Beheneth-20 and | — | — | — | — | — | — |
| | | tocopherol | | | | | | |
| 9 | | Steareth-20 | — | — | — | — | — | — |
| 10 | | PEG-100 stearate | — | — | — | — | — | — |
| 11 | | PEG-60 glyceryl | 1.0 | — | — | — | — | — |
| | | isostearate and | | | | | | |
| | | tocopherol | | | | | | |
| 12 | | Polysorbate 60 | — | 1.0 | — | — | — | — |
| 13 | | Glyceryl stearate | — | — | 1.0 | — | — | — |
| 14 | | Sorbitan | — | — | — | 1.0 | — | — |
| | | isostearate | | | | | | |
| 15 | | Sucrose stearate | — | — | — | — | 1.0 | — |
| | | and sucrose | | | | | | |
| | | palmitate | | | | | | |
| 16 | | Polyglyceryl-10 | — | — | — | — | — | 1.0 |
| | | laurate | | | | | | |
| 17 | Catalyst | Mixture¹⁾ of | 1.5 | 1.5 | 1.5 | 1.5 | 1.5 | 1.5 |
| | | vinyl | | | | | | |
| | | dimethicone, | | | | | | |
| | | divinyl | | | | | | |
| | | disiloxane, and | | | | | | |
| | | platinum catalyst | | | | | | |
| 18 | pH adjuster | Potassium | q.s. | q.s. | q.s. | q.s. | q.s. | q.s. |
| | | hydroxide | | | | | | |
| 19 | Lower alcohol | Denatured | 10.0 | 10.0 | 10.0 | 10.0 | 10.0 | 10.0 |
| | | alcohol | | | | | | |
| 20 | Preservative | Phenoxyethanol | 0.5 | 0.5 | 0.5 | 0.5 | 0.5 | 0.5 |
| 21 | Filler | Nylon-12 | 8.0 | 8.0 | 8.0 | 8.0 | 8.0 | 8.0 |
| | | particles, average | | | | | | |
| | | particle size: 5 μm | | | | | | |

| Total | | 100 | 100 | 100 | 100 | 100 | 100 |
|---|---|---|---|---|---|---|---|
| Blending | (i) Oil content (part by mass) excluding | 0.045 | 0.045 | 0.045 | 0.045 | 0.045 | 0.045 |
| ratio of | reactive polymer (vinyl dimethicone) | | | | | | |
| catalyst | (ii) Platinum catalyst content (part by | 0.030 | 0.030 | 0.030 | 0.030 | 0.030 | 0.030 |
| | mass) | | | | | | |
| | Blending ratio (%) of catalyst to oil: | 67 | 67 | 67 | 67 | 67 | 67 |
| | {(ii) × 100}/(i) | | | | | | |
| Evaluation | Cross-linking properties of film | A | A | A | A | A | A |
| | Appearance of film | A | A | A | A | A | A |
| | Abrasion resistance (number of repetition) | C(8) | B(11) | B(10) | C(7) | C(8) | C(9) |
| | Emulsifiability | A | A | A | A | A | A |

¹⁾Divinyl disiloxane in the mixture is considered as an oil in the calculation of the blending ratio of catalysts to oils. The ratios of the ingredients in the mixture: 2% by mass of platinum, 3% by mass of divinyl disiloxane, and 95% by mass of vinyl dimethicone.

<Results>

As can be seen from the results in Table 3, even in cases where various emulsifiers are used, each oil-in-water formulation as the second agent can shorten the cross-linking reaction time of the film and also improve the durability of the film.

### Test Example 3: Determination of Cross-Linking Properties, Durability, and the Like of Films in Cases of Use of High-Molecular-Weight Emulsifier as Emulsifier in Second Agent

In Test Example 3, the cross-linking properties, durability, and the like were investigated for cases where a high-molecular-weight emulsifier was used as an emulsifier in the second agent in the coating-type body corrective film formation agent. The results are shown in Table 4.

<Method of Producing First Agent Used with Second Agent>

A first agent was prepared by uniformly mixing 45 parts by mass of divinyl dimethicone at 165,000 cSt as a second unsaturated organopolysiloxane, 10 parts by mass of hydrogen dimethicone at 50 cSt as a second hydride functionalized polysiloxane, 10 parts by mass of zinc oxide, and 35 parts by mass of dimethicone at 1.5 cSt as an oil.

<Method of Producing Second Agents as Oil-In-Water Compositions>

Using the formulations shown in Table 4, second agents as oil-in-water compositions were produced by the following method. Here, the numbers shown below correspond to the numbers representing the ingredients on the left side of the formulations in Table 4.

### Example 21

The materials of Nos. 1 to 3 and 13 to 16 were uniformly mixed to prepare an aqueous-phase part, and the catalyst of No. 12 was then added thereto, followed by uniformly mixing the resulting mixture to prepare a second agent as an oil-in-water composition of Example 21.

### Examples 22 to 33

Second agents as oil-in-water compositions of Examples 22 to 33 were prepared in the same manner as in Example 21 except that their formulations in Table 4 were used. The high-molecular-weight emulsifiers of Nos. 4 to 11 were blended when the aqueous-phase parts were prepared.

TABLE 4

| | Formulation of second agent | | Oil-in-water | | | | | | |
|---|---|---|---|---|---|---|---|---|---|
| No. | Ingredient | Ingredient name | Ex. 21 | Ex. 22 | Ex. 23 | Ex. 24 | Ex. 25 | Ex. 26 | Ex. 27 |
| 1 | Water | Ion exchanged water | Balance | Balance | Balance | Balance | Balance | Balance | Balance |
| 2 | Moisturizing agent | Glycerol | 25.0 | 25.0 | 25.0 | 25.0 | 25.0 | 25.0 | 25.0 |
| 3 | Thickener | Carboxyvinyl polymer | 0.2 | 0.2 | 0.2 | 0.2 | 0.2 | 0.2 | 0.2 |
| 4 | High-molecular- | Acrylates/C10-30 alkyl | — | 0.1 | 0.1 | 1.0 | 2.0 | 3.0 | — |
| | weight emulsifier | acrylate crosspolymer¹⁾ | | | | | | | |
| 5 | | Acrylates/C10-30 alkyl | — | — | — | — | — | — | 0.1 |
| | | acrylate crosspolymer²⁾ | | | | | | | |
| 6 | | Acrylates/C10-30 alkyl | — | — | — | — | — | — | — |
| | | acrylate crosspolymer³⁾ | | | | | | | |
| 7 | | Hydroxypropyl methylcellulose | — | — | — | — | — | — | — |
| | | stearoxy ether | | | | | | | |
| 8 | | Polyurethane-59, butylene | — | — | — | — | — | — | — |
| | | glycol, water, and tocopherol | | | | | | | |
| 9 | | PEG-240/HDI copolymer bis- | — | — | — | — | — | — | — |
| | | decyltetradeceth-20 ether | | | | | | | |
| | | and dibutyl hydroxytoluene | | | | | | | |
| 10 | | Ammonium | — | — | — | — | — | — | — |
| | | acryloyldimethyltaurate/ | | | | | | | |
| | | VP copolymer | | | | | | | |
| 11 | | Ammonium | — | — | — | — | — | — | — |
| | | acryloyldimethyltaurate/ | | | | | | | |
| | | beheneth-25 methacrylate | | | | | | | |
| | | crosspolymer | | | | | | | |
| 12 | Catalyst | Mixture⁴⁾ of vinyl | 1.3 | 1.3 | 1.3 | 1.3 | 1.3 | 1.3 | 1.3 |
| | | dimethicone, divinyl | | | | | | | |
| | | disiloxane, and | | | | | | | |
| | | platinum catalyst | | | | | | | |
| 13 | pH adjuster | Potassium hydroxide | q.s. | q.s. | q.s. | q.s. | q.s. | q.s. | q.s. |
| 14 | Lower alcohol | Denatured alcohol | 10.0 | 10.0 | 10.0 | 10.0 | 10.0 | 10.0 | 10.0 |
| 15 | Preservative | Phenoxyethanol | 0.5 | 0.5 | 0.5 | 0.5 | 0.5 | 0.5 | 0.5 |
| 16 | Filler | Methyl methacrylate | 8.0 | 8.0 | 8.0 | 8.0 | 8.0 | 8.0 | 8.0 |
| | | crosspolymer silica | | | | | | | |
| | | and silica | | | | | | | |

| Total | | 100 | 100 | 100 | 100 | 100 | 100 | 100 |
|---|---|---|---|---|---|---|---|---|
| Blending | (i) Oil content (part by mass) excluding | 0.039 | 0.039 | 0.039 | 0.039 | 0.039 | 0.039 | 0.039 |
| ratio of | reactive polymer (vinyl dimethicone) | | | | | | | |
| catalyst | (ii) Platinum catalyst content (part by | 0.026 | 0.026 | 0.026 | 0.026 | 0.026 | 0.026 | 0.026 |
| | mass) | | | | | | | |
| | Blending ratio (%) of catalyst to oil: | 67 | 67 | 67 | 67 | 67 | 67 | 67 |
| | {(ii) × 100}/(i) | | | | | | | |
| Evaluation | Cross-linking properties of film | A | A | A | A | B | C | A |
| | Appearance of film | A | A | A | A | A | B | A |
| | Abrasion resistance (number of repetition) | A(50) | A(27) | A(25) | A(20) | B(17) | C(8) | A(23) |
| | Emulsifiability | C | B | A | A | A | A | A |

| | Formulation of second agent | | Oil-in-water | | | | | |
|---|---|---|---|---|---|---|---|---|
| No. | Ingredient | Ingredient name | Ex. 28 | Ex. 29 | Ex. 30 | Ex. 31 | Ex. 32 | Ex. 33 |
| 1 | Water | Ion exchanged water | Balance | Balance | Balance | Balance | Balance | Balance |
| 2 | Moisturizing agent | Glycerol | 25.0 | 25.0 | 25.0 | 25.0 | 25.0 | 25.0 |
| 3 | Thickener | Carboxyvinyl polymer | 0.2 | 0.2 | 0.2 | 0.2 | 0.2 | 0.2 |
| 4 | High-molecular- | Acrylates/C10-30 alkyl | — | — | — | — | — | — |
| | weight emulsifier | acrylate crosspolymer¹⁾ | | | | | | |
| 5 | | Acrylates/C10-30 alkyl | — | — | — | — | — | — |
| | | acrylate crosspolymer²⁾ | | | | | | |
| 6 | | Acrylates/C10-30 alkyl | 0.1 | — | — | — | — | — |
| | | acrylate crosspolymer³⁾ | | | | | | |
| 7 | | Hydroxypropyl methylcellulose | — | 0.1 | — | — | — | — |
| | | stearoxy ether | | | | | | |
| 8 | | Polyurethane-59, butylene | — | — | 1.0 | — | — | — |
| | | glycol, water, and tocopherol | | | | | | |
| 9 | | PEG-240/HDI copolymer bis- | — | — | — | 1.0 | — | — |
| | | decyltetradeceth-20 ether | | | | | | |
| | | and dibutyl hydroxytoluene | | | | | | |
| 10 | | Ammonium | — | — | — | — | 1.0 | — |
| | | acryloyldimethyltaurate/ | | | | | | |
| | | VP copolymer | | | | | | |
| 11 | | Ammonium | — | — | — | — | — | 1.0 |
| | | acryloyldimethyltaurate/ | | | | | | |
| | | beheneth-25 methacrylate | | | | | | |
| | | crosspolymer | | | | | | |
| 12 | Catalyst | Mixture⁴⁾ of vinyl | 1.3 | 1.3 | 1.3 | 1.3 | 1.3 | 1.3 |
| | | dimethicone, divinyl | | | | | | |
| | | disiloxane, and | | | | | | |
| | | platinum catalyst | | | | | | |
| 13 | pH adjuster | Potassium hydroxide | q.s. | q.s. | q.s. | q.s. | q.s. | q.s. |
| 14 | Lower alcohol | Denatured alcohol | 10.0 | 10.0 | 10.0 | 10.0 | 10.0 | 10.0 |
| 15 | Preservative | Phenoxyethanol | 0.5 | 0.5 | 0.5 | 0.5 | 0.5 | 0.5 |
| 16 | Filler | Methyl methacrylate | 8.0 | 8.0 | 8.0 | 8.0 | 8.0 | 8.0 |
| | | crosspolymer silica | | | | | | |
| | | and silica | | | | | | |

| Total | | 100 | 100 | 100 | 100 | 100 | 100 |
|---|---|---|---|---|---|---|---|
| Blending | (i) Oil content (part by mass) excluding | 0.039 | 0.039 | 0.039 | 0.039 | 0.039 | 0.039 |
| ratio of | reactive polymer (vinyl dimethicone) | | | | | | |
| catalyst | (ii) Platinum catalyst content (part by | 0.026 | 0.026 | 0.026 | 0.026 | 0.026 | 0.026 |
| | mass) | | | | | | |
| | Blending ratio (%) of catalyst to oil: | 67 | 67 | 67 | 67 | 67 | 67 |
| | {(ii) × 100}/(i) | | | | | | |
| Evaluation | Cross-linking properties of film | A | A | A | A | A | A |
| | Appearance of film | A | A | A | A | A | A |
| | Abrasion resistance (number of repetition) | A(45) | A(26) | B(16) | B(17) | B(14) | B(19) |
| | Emulsifiability | A | A | A | B | A | A |

¹⁾PEMULEN ™ TR-2 (Iwase Cosfa Co., Ltd.)
²⁾PEMULEN ™ TR-1 POLYMER (Iwase Cosfa Co., Ltd.)
³⁾PEMULEN ™ EZ-4U POLYMER (Iwase Cosfa Co., Ltd.)
⁴⁾Divinyl disiloxane in the mixture is considered as an oil in the calculation of the blending ratio of catalysts to oils. The ratios of the ingredients in the mixture: 2% by mass of platinum, 3% by mass of divinyl disiloxane, and 95% by mass of vinyl dimethicone.

<Results>

As can be seen from the results in Table 4, even in cases where various high-molecular-weight emulsifiers are used, each oil-in-water formulation as the second agent can shorten the cross-linking reaction time of the film and also improve the durability of the film.

Comparison between Examples 21 and 22 shows that use of a high-molecular-weight emulsifier can improve the emulsifiability. Comparison among Examples 24 to 26 shows that the blending amount of the high-molecular-weight emulsifier is preferably 2.0% by mass or less in view of film durability.

### Test Example 4: Determination of Cross-Linking Properties, Durability, and the Like of Films with Different Blending Ratios of Catalyst to Oil in Second Agent

In Test Example 4, the cross-linking properties, durability, and the like of films with different blending ratios of the catalyst to the oil in the second agent were investigated. The results are shown in Table 5.

<Method of Producing First Agent Used with Second Agent>

A first agent was prepared by uniformly mixing 45 parts by mass of divinyl dimethicone at 165,000 cSt as a second unsaturated organopolysiloxane, 10 parts by mass of hydrogen dimethicone at 45 cSt as a second hydride functionalized polysiloxane, 7.5 parts by mass of silica silylate as a filler, 37.5 parts by mass of a mixture of dimethicone and trisiloxane as an oil.

<Method of Producing Second Agents as Oil-In-Water Compositions>

Using the formulations shown in Table 5, second agents as oil-in-water compositions were produced by the following method. Here, the numbers shown below correspond to the numbers representing the ingredients on the left side of the formulations in Table 5.

### Example 34

The materials of Nos. 1 to 6, 13, and 14 were uniformly mixed to prepare an aqueous-phase part.

The materials of Nos. 7 and 9 were uniformly mixed to prepare an oil-phase part.

The oil-phase part was added to the aqueous-phase part, and the resulting mixture was uniformly mixed to prepare a second agent as an oil-in-water composition of Example 34.

### Examples 35 to 48

Second agents as oil-in-water compositions of Examples 35 to 48 were prepared in the same manner as in Example 34 except that their formulations in Table 5 were used.

TABLE 5

| | Formulation of second agent | | Oil-in-water | | | | | | | | |
|---|---|---|---|---|---|---|---|---|---|---|---|
| No. | Ingredient | Ingredient name | Ex. 34 | Ex. 35 | Ex. 36 | Ex. 37 | Ex. 38 | Ex. 39 | Ex. 40 | Ex. 41 | Ex. 42 |
| 1 | Water | Ion exchanged | Balance | Balance | Balance | Balance | Balance | Balance | Balance | Balance | Balance |
| | | water | | | | | | | | | |
| 2 | Polyhydric alcohol | 1,3-butylene | 5.0 | 5.0 | 5.0 | 5.0 | 5.0 | 5.0 | 5.0 | 5.0 | 5.0 |
| | | glycol | | | | | | | | | |
| 3 | Moisturizing agent | Glycerol | 5.0 | 5.0 | 5.0 | 5.0 | 5.0 | 5.0 | 5.0 | 5.0 | 5.0 |
| 4 | Thickener | Carboxyvinyl | 0.1 | 0.1 | 0.1 | 0.1 | 0.1 | 0.1 | 0.1 | 0.1 | 0.1 |
| | | polymer | | | | | | | | | |
| 5 | High-molecular- | Acrylates/C10- | 0.2 | 0.2 | 0.2 | 0.2 | 0.2 | 0.2 | 0.2 | 0.2 | 0.2 |
| | weight emulsifier | 30 alkyl acrylate | | | | | | | | | |
| | | crosspolymer¹⁾ | | | | | | | | | |
| 6 | Emulsifier | PEG-12 | 1.0 | 1.0 | 1.0 | 1.0 | 1.0 | 1.0 | 1.0 | 1.0 | 1.0 |
| | | dimethicone | | | | | | | | | |
| 7 | Catalyst | Mixture²⁾ of | 1.8 | 1.8 | 1.8 | 1.8 | 1.8 | 1.8 | 1.8 | 1.8 | 1.8 |
| | | vinyl | | | | | | | | | |
| | | dimethicone, | | | | | | | | | |
| | | divinyl | | | | | | | | | |
| | | disiloxane, and | | | | | | | | | |
| | | platinum catalyst | | | | | | | | | |
| 8 | First unsaturated | Vinyl | — | 3.0 | 5.0 | 10.0 | — | 8.0 | — | 8.0 | — |
| | organopolysiloxane | dimethicone | | | | | | | | | |
| | | (viscosity: 250 | | | | | | | | | |
| | | cSt) | | | | | | | | | |
| 9 | Oil | Dimethicone, | 8.0 | 8.0 | 8.0 | 8.0 | 8.0 | 8.0 | 8.0 | 8.0 | 8.0 |
| | | polysilicone-11, | | | | | | | | | |
| | | butylene glycol, | | | | | | | | | |
| | | water, and decyl | | | | | | | | | |
| | | glucoside (oil | | | | | | | | | |
| | | content: 89% by | | | | | | | | | |
| | | mass) | | | | | | | | | |
| 10 | | Dimethicone | — | — | — | — | — | — | 20.0 | 20.0 | — |
| | | (viscosity: 1.5 | | | | | | | | | |
| | | cSt) | | | | | | | | | |
| 11 | | Diisopropyl | — | — | — | — | 10.0 | 10.0 | 10.0 | 10.0 | 10.0 |
| | | sebacate | | | | | | | | | |
| 12 | | Coconut alkane | — | — | — | — | — | — | — | — | 20.0 |
| | | and coco- | | | | | | | | | |
| | | caprylate/caprate | | | | | | | | | |
| 13 | Lower alcohol | Water-containing | 10.0 | 10.0 | 10.0 | 10.0 | 10.0 | 10.0 | 10.0 | 10.0 | 10.0 |
| | | ethanol | | | | | | | | | |
| 14 | Preservative | Phenoxyethanol | 0.5 | 0.5 | 0.5 | 0.5 | 0.5 | 0.5 | 0.5 | 0.5 | 0.5 |

| Total | | 100 | 100 | 100 | 100 | 100 | 100 | 100 | 100 | 100 |
|---|---|---|---|---|---|---|---|---|---|---|
| Blending | (i) Oil content (part by mass) | 7.2 | 7.2 | 7.2 | 7.2 | 17.2 | 17.2 | 37.2 | 37.2 | 37.2 |
| ratio of | excluding reactive polymer (vinyl | | | | | | | | | |
| catalyst | dimethicone) | | | | | | | | | |
| | (ii) Platinum catalyst content (part by | 0.036 | 0.036 | 0.036 | 0.036 | 0.036 | 0.036 | 0.036 | 0.036 | 0.036 |
| | mass) | | | | | | | | | |
| | Blending ratio (%) of catalyst to oil: | 0.50 | 0.50 | 0.50 | 0.50 | 0.21 | 0.21 | 0.10 | 0.10 | 0.10 |
| | {(ii) × 100}/(i) | | | | | | | | | |
| Evaluation | Cross-linking properties of film | A | A | A | A | A | A | A | A | A |
| | Appearance of film | B | A | A | A | B | A | B | A | B |
| | Abrasion resistance (number of | B(10) | B(12) | A(36) | A(40) | C(9) | A(28) | C(8) | A(25) | C(9) |
| | repetition) | | | | | | | | | |
| | Emulsifiability | A | A | A | A | A | A | A | A | A |

| | Formulation of second agent | | Oil-in-water | | | | | |
|---|---|---|---|---|---|---|---|---|
| No. | Ingredient | Ingredient name | Ex. 43 | Ex. 44 | Ex. 45 | Ex. 46 | Ex. 47 | Ex. 48 |
| 1 | Water | Ion exchanged | Balance | Balance | Balance | Balance | Balance | Balance |
| | | water | | | | | | |
| 2 | Polyhydric alcohol | 1,3-butylene | 5.0 | 5.0 | 5.0 | 5.0 | 5.0 | 5.0 |
| | | glycol | | | | | | |
| 3 | Moisturizing agent | Glycerol | 5.0 | 5.0 | 5.0 | 5.0 | 5.0 | 5.0 |
| 4 | Thickener | Carboxyvinyl | 0.1 | 0.1 | 0.1 | 0.1 | 0.1 | 0.1 |
| | | polymer | | | | | | |
| 5 | High-molecular- | Acrylates/C10- | 0.2 | 0.2 | 0.2 | 0.2 | 0.2 | 0.2 |
| | weight emulsifier | 30 alkyl acrylate | | | | | | |
| | | crosspolymer¹⁾ | | | | | | |
| 6 | Emulsifier | PEG-12 | 1.0 | 1.0 | 1.0 | 1.0 | 1.0 | 1.0 |
| | | dimethicone | | | | | | |
| 7 | Catalyst | Mixture²⁾ of | 1.8 | 1.8 | 1.8 | 1.8 | 1.8 | 1.8 |
| | | vinyl | | | | | | |
| | | dimethicone, | | | | | | |
| | | divinyl | | | | | | |
| | | disiloxane, and | | | | | | |
| | | platinum catalyst | | | | | | |
| 8 | First unsaturated | Vinyl | 8.0 | — | 8.0 | — | — | — |
| | organopolysiloxane | dimethicone | | | | | | |
| | | (viscosity: 250 | | | | | | |
| | | cSt) | | | | | | |
| 9 | Oil | Dimethicone, | 8.0 | 8.0 | 8.0 | 8.0 | 8.0 | 8.0 |
| | | polysilicone-11, | | | | | | |
| | | butylene glycol, | | | | | | |
| | | water, and decyl | | | | | | |
| | | glucoside (oil | | | | | | |
| | | content: 89% by | | | | | | |
| | | mass) | | | | | | |
| 10 | | Dimethicone | — | — | — | 20.0 | — | — |
| | | (viscosity: 1.5 | | | | | | |
| | | cSt) | | | | | | |
| 11 | | Diisopropyl | 10.0 | 10.0 | 10.0 | 20.0 | 20.0 | 20.0 |
| | | sebacate | | | | | | |
| 12 | | Coconut alkane | 20.0 | 25.0 | 25.0 | — | 20.0 | 30.0 |
| | | and coco- | | | | | | |
| | | caprylate/caprate | | | | | | |
| 13 | Lower alcohol | Water-containing | 10.0 | 10.0 | 10.0 | 10.0 | 10.0 | 10.0 |
| | | ethanol | | | | | | |
| 14 | Preservative | Phenoxyethanol | 0.5 | 0.5 | 0.5 | 0.5 | 0.5 | 0.5 |

| Total | | 100 | 100 | 100 | 100 | 100 | 100 |
|---|---|---|---|---|---|---|---|
| Blending | (i) Oil content (part by mass) | 37.2 | 32.2 | 32.2 | 47.2 | 47.2 | 57.2 |
| ratio of | excluding reactive polymer (vinyl | | | | | | |
| catalyst | dimethicone) | | | | | | |
| | (ii) Platinum catalyst content (part by | 0.036 | 0.036 | 0.036 | 0.036 | 0.036 | 0.036 |
| | mass) | | | | | | |
| | Blending ratio (%) of catalyst to oil: | 0.10 | 0.11 | 0.11 | 0.076 | 0.076 | 0.063 |
| | {(ii) × 100}/(i) | | | | | | |
| Evaluation | Cross-linking properties of film | A | A | A | B | B | C |
| | Appearance of film | A | B | A | B | B | B |
| | Abrasion resistance (number of | A(31) | C(7) | A(28) | C(5) | C(6) | C(5) |
| | repetition) | | | | | | |
| | Emulsifiability | A | A | A | B | B | B |

¹⁾PEMULEN ™ EZ-4U POLYMER (Iwase Cosfa Co., Ltd.)
²⁾Divinyl disiloxane in the mixture is considered as an oil in the calculation of the blending ratio of catalysts to oils. The ratios of the ingredients in the mixture: 2% by mass of platinum, 4% by mass of divinyl disiloxane, and 94% by mass of vinyl dimethicone.

<Results>

As can be seen from the results in Table 5, a blending ratio of the catalyst to the oil of 0.060% or more can further shorten the cross-linking reaction time of the film and also further improve the durability of the film.

«Formulation Examples of Coating-Type Body Corrective Film Formation Agents»

The following are formulation examples of first agents and second agents that can be used as coating-type body corrective film formation agents of the present disclosure, but the coating-type body corrective film formation agent of the present invention is not limited to these examples. All the coating-type body corrective film formation agents containing the first agents and the second agents described in the following formulation examples successfully shortened the cross-linking reaction time in the film formation and improved the durability of the obtained films. Also for the formulation examples shown below, the tests described above were performed. The results are summarized in Tables 6 to 8. Here, “100<” represents cases in the abrasion resistance test where the number of repetition until the occurrence of damage to the film exceeded 100 times.

### Formulation Example A

(First Agent)

A first agent was prepared by uniformly mixing 20 parts by mass of divinyl dimethicone at 165,000 cSt as a second unsaturated organopolysiloxane, 10 parts by mass of hydrogen dimethicone at 45 cSt as a second hydride functionalized polysiloxane, 30 parts by mass of ion exchanged water, 10 parts by mass of sodium chloride, 1 part by mass of 1,3-butylene glycol, 0.5 parts by mass of phenoxyethanol, and 28.5 parts by mass of a mixture of dimethicone and trisiloxane as an oil.

(Second Agent)

Using the formulations shown in Table 6, second agents as oil-in-water compositions were produced by the following method. Here, the numbers shown below correspond to the numbers representing the ingredients on the left side of the formulations in Table 6.

### a. Formulation Example 1

The materials of Nos. 1 to 5 and 9 to 12 were uniformly mixed to prepare an aqueous-phase part, and the catalyst of No. 6 was then added thereto, followed by uniformly mixing the resulting mixture to prepare a second agent as an oil-in-water composition of Formulation Example 1.

### b. Formulation Examples 2 to 5

Second agents as oil-in-water compositions of Formulation Examples 2 to 5 were prepared in the same manner as in Formulation Example 1 except that their formulations in Table 6 were used. The oils of Nos. 7 and 8 were mixed with the catalyst of No. 6, and the mixture as an oil-phase part was added to an aqueous-phase part.

TABLE 6

| | | | Form. | Form. | Form. | Form. | Form. |
|---|---|---|---|---|---|---|---|
| No. | Ingredient | Ingredient name | Ex. 1 | Ex. 2 | Ex. 3 | Ex. 4 | Ex. 5 |
| 1 | Water | Ion exchanged water | Balance | Balance | Balance | Balance | Balance |
| 2 | Moisturizing agent | Glycerol | 30.0 | 30.0 | 30.0 | 30.0 | 30.0 |
| 3 | | PPG-20 methyl | 5.0 | 5.0 | 5.0 | 5.0 | 5.0 |
| | | glucose ether | | | | | |
| 4 | Thickener | Carboxyvinyl | 0.2 | 0.2 | 0.2 | 0.2 | 0.2 |
| | | polymer | | | | | |
| 5 | High-molecular- | alkyl acrylate | 0.1 | 0.1 | 0.1 | 0.1 | 0.1 |
| | weight emulsifier | Acrylates/C10-30 | | | | | |
| | | crosspolymer¹⁾ | | | | | |
| 6 | Catalyst | Mixture²⁾ of vinyl | 1.0 | 1.0 | 1.0 | 1.0 | 1.0 |
| | | dimethicone, divinyl | | | | | |
| | | disiloxane, and | | | | | |
| | | platinum catalyst | | | | | |
| 7 | Oil | Triethylhexanoin | — | 10.0 | 15.0 | — | 10.0 |
| 8 | | Squalane and | — | — | — | 15.0 | 10.0 |
| | | tocopherol | | | | | |
| 9 | pH adjuster | Potassium hydroxide | q.s. | q.s. | q.s. | q.s. | q.s. |
| 10 | Lower alcohol | Denatured alcohol | 10.0 | 10.0 | 10.0 | 10.0 | 10.0 |
| 11 | Preservative | Phenoxyethanol | 0.5 | 0.5 | 0.5 | 0.5 | 0.5 |
| 12 | Filler | Nylon-12 particles, | 8.0 | 8.0 | 8.0 | 8.0 | 8.0 |
| | | average particle size: | | | | | |
| | | 5 μm | | | | | |

| Total | | 100 | 100 | 100 | 100 | 100 |
|---|---|---|---|---|---|---|
| Blending | (i) Oil content (part by mass) excluding | 0.040 | 10.0 | 15.0 | 15.0 | 20.0 |
| ratio of | reactive polymer (vinyl dimethicone) | | | | | |
| catalyst | (ii) Platinum catalyst content (part by | 0.020 | 0.020 | 0.020 | 0.020 | 0.020 |
| | mass) | | | | | |
| | Blending ratio (%) of catalyst to oil: | 50 | 0.20 | 0.13 | 0.13 | 0.10 |
| | {(ii) × 100}/(i) | | | | | |
| Evaluation | Cross-linking properties of film | A | A | A | A | A |
| | Appearance of film | A | A | A | A | A |
| | Abrasion resistance (number of repetition) | A(75) | A(60) | A(55) | A(54) | A(40) |
| | Emulsifiability | A | A | A | A | A |

¹⁾PEMULEN ™ TR-2 (Iwase Cosfa Co., Ltd.)
²⁾Divinyl disiloxane in the mixture is considered as an oil in the calculation of the blending ratio of catalysts to oils. The ratios of the ingredients in the mixture: 2% by mass of platinum, 4% by mass of divinyl disiloxane, and 94% by mass of vinyl dimethicone.

### Formulation Example B

(First Agent)

A first agent was prepared by uniformly mixing 42 parts by mass of divinyl dimethicone at 165,000 cSt as a second unsaturated organopolysiloxane, 10 parts by mass of hydrogen dimethicone at 50 cSt as a second hydride functionalized polysiloxane, 10 parts by mass of graphene as a filler, and 38 parts by mass of dimethicone as an oil.

(Second Agent)

Using the formulations shown in Table 7, second agents as oil-in-water compositions were produced by the following method. Here, the numbers shown below correspond to the numbers representing the ingredients on the left side of the formulations in Table 7.

### a. Formulation Example 6

The materials of Nos. 1 to 3, 5, 6, 9, and 14 to 16 were uniformly mixed to prepare an aqueous-phase part, and the catalyst of No. 8 was then added thereto, followed by uniformly mixing the resulting mixture to prepare a second agent as an oil-in-water composition of Formulation Example 6.

### b. Formulation Examples 7 to 10

Second agents as oil-in-water compositions of Formulation Examples 7 to 10 were prepared in the same manner as in Formulation Example 6 except that their formulations in Table 7 were used. The materials of Nos. 11 to 13 were mixed with the catalyst of No. 8, and the mixture as an oil-phase part was added to an aqueous-phase part.

TABLE 7

| | | | Form. | Form. | Form. | Form. | Form. |
|---|---|---|---|---|---|---|---|
| No. | Ingredient | Ingredient name | Ex. 6 | Ex. 7 | Ex. 8 | Ex. 9 | Ex. 10 |
| 1 | Water | Ion exchanged water | Balance | Balance | Balance | Balance | Balance |
| 2 | Moisturizing agent | Glycerol | 25.0 | 25.0 | 25.0 | 25.0 | 25.0 |
| 3 | | PPG-20 methyl | 5.0 | — | — | — | — |
| | | glucose ether | | | | | |
| 4 | Emollient | PPG-20 methyl | — | 10.0 | 10.0 | 10.0 | 10.0 |
| | | glucose ether | | | | | |
| | | distearate | | | | | |
| 5 | Thickener | Carboxyvinyl | 0.2 | — | — | — | — |
| | | polymer | | | | | |
| 6 | High-molecular- | Acrylates/C10-30 | 0.1 | — | — | — | — |
| | weight emulsifier | alkyl acrylate | | | | | |
| | | crosspolymer¹⁾ | | | | | |
| 7 | | Acrylates/C10-30 | — | 0.3 | 0.3 | 0.3 | 0.3 |
| | | alkyl acrylate | | | | | |
| | | crosspolymer²⁾ | | | | | |
| 8 | Catalyst | Mixture³⁾ of vinyl | 1.2 | 1.2 | 1.2 | 1.2 | 1.2 |
| | | dimethicone, divinyl | | | | | |
| | | disiloxane, and | | | | | |
| | | platinum catalyst | | | | | |
| 9 | pH adjuster | Potassium hydroxide | q.s. | q.s. | q.s. | q.s. | q.s. |
| 10 | Emulsifier | PEG-12 dimethicone | | 1.0 | 1.0 | 1.0 | 1.0 |
| 11 | First unsaturated | Vinyl dimethicone | — | — | 5.0 | 5.0 | 5.0 |
| | organopolysiloxane | (viscosity: 250 cSt) | | | | | |
| 12 | Oil | Diisopropyl sebacate | — | — | — | 10.0 | 15.0 |
| 13 | | Dimethicone, | — | 5.0 | 5.0 | 5.0 | 5.0 |
| | | polysilicone-11, | | | | | |
| | | butylene glycol, | | | | | |
| | | water, and decyl | | | | | |
| | | glucoside (oil | | | | | |
| | | content: 89% by | | | | | |
| | | mass) | | | | | |
| 14 | Lower alcohol | Denatured alcohol | 10.0 | 10.0 | 10.0 | 10.0 | 10.0 |
| 15 | Preservative | Phenoxyethanol | 0.5 | 0.5 | 0.5 | 0.5 | 0.5 |
| 16 | Filler | Nylon-12 particles, | 8.0 | 8.0 | 8.0 | 8.0 | 8.0 |
| | | average particle size: | | | | | |
| | | 5 μm | | | | | |

| Total | | 100 | 100 | 100 | 100 | 100 |
|---|---|---|---|---|---|---|
| Blending | (i) Oil content (part by mass) excluding | 0.048 | 4.5 | 4.5 | 14.5 | 19.5 |
| ratio of | reactive polymer (vinyl dimethicone) | | | | | |
| catalyst | (ii) Platinum catalyst content (part by | 0.024 | 0.024 | 0.024 | 0.024 | 0.024 |
| | mass) | | | | | |
| | Blending ratio (%) of catalyst to oil: | 50 | 0.53 | 0.53 | 0.17 | 0.12 |
| | {(ii) × 100}/(i) | | | | | |
| Evaluation | Cross-linking properties of film | A | A | A | A | A |
| | Appearance of film | A | A | A | A | A |
| | Abrasion resistance (number of repetition) | A(100<) | A(100<) | A(100<) | A(80) | A(70) |
| | Emulsifiability | A | A | A | A | A |

¹⁾PEMULEN ™ TR-2 (Iwase Cosfa Co., Ltd.)
²⁾PEMULEN ™ EZ-4U POLYMER (Iwase Cosfa Co., Ltd.)
³⁾Divinyl disiloxane in the mixture is considered as an oil in the calculation of the blending ratio of catalysts to oils. The ratios of the ingredients in the mixture: 2% by mass of platinum, 4% by mass of divinyl disiloxane, and 94% by mass of vinyl dimethicone.

### Formulation Example C

(First Agent)

A first agent was prepared by uniformly mixing 30 parts by mass of divinyl dimethicone at 165,000 cSt as a second unsaturated organopolysiloxane, 7 parts by mass of hydrogen dimethicone at 45 cSt as a second hydride functionalized polysiloxane, 6 parts by mass of silica silylate as a filler, and 57 parts by mass of a mixture of dimethicone and trisiloxane as an oil.

(Second Agent)

Using the formulations shown in Table 8, second agents as oil-in-water compositions were produced by the following method. Here, the numbers shown below correspond to the numbers representing the ingredients on the left side of the formulations in Table 8.

### a. Formulation Example 11

The materials of Nos. 1 to 5, 7, 8, and 12 to 14 were uniformly mixed to prepare an aqueous-phase part.

The materials of Nos. 6, and 9 to 11 were uniformly mixed to prepare an oil-phase part.

The oil-phase part was added to the aqueous-phase part, and the resulting mixture was uniformly mixed to prepare a second agent as an oil-in-water composition of Formulation Example 11.

### b. Formulation Examples 12 and 13

Second agents as oil-in-water compositions of Formulation Examples 12 and 13 were prepared in the same manner as in Formulation Example 11 except that their formulations in Table 8 were used.

TABLE 8

| | | | Form. | Form. | Form. |
|---|---|---|---|---|---|
| No. | Ingredient | Ingredient name | Ex. 11 | Ex. 12 | Ex. 13 |
| 1 | Water | Ion exchanged water | Balance | Balance | Balance |
| 2 | Moisturizing agent | Glycerol | 10.0 | 10.0 | 10.0 |
| 3 | Emollient | PPG-20 methyl glucose | 10.0 | 10.0 | 10.0 |
| | | ether distearate | | | |
| 4 | Thickener | Carboxyvinyl polymer | 0.2 | 0.2 | 0.2 |
| 5 | High-molecular- | Acrylates/C10-30 alkyl | 0.5 | 0.5 | 0.5 |
| | weight emulsifier | acrylate crosspolymer¹⁾ | | | |
| 6 | Catalyst | Mixture of vinyl dimethicone, | 1.2 | 1.2 | 1.2 |
| | | divinyl disiloxane, and | | | |
| | | platinum catalyst²⁾ | | | |
| 7 | pH adjuster | Potassium hydroxide | q.s. | q.s. | q.s. |
| 8 | Emulsifier | PEG-12 dimethicone | 1.0 | 1.0 | 1.0 |
| 9 | First unsaturated | Vinyl dimethicone | 5.0 | 5.0 | 5.0 |
| | organopolysiloxane | (viscosity: 250 cSt) | | | |
| 10 | Oil | Diisopropyl sebacate | 10.0 | 10.0 | 10.0 |
| 11 | | Dimethicone, polysilicone-11, | 5.0 | 5.0 | 5.0 |
| | | butylene glycol, water, and | | | |
| | | decyl glucoside (oil content: | | | |
| | | 89% by mass) | | | |
| 12 | Lower alcohol | Denatured alcohol | 10.0 | 10.0 | 10.0 |
| 13 | Preservative | Phenoxyethanol | 0.5 | 0.5 | 0.5 |
| 14 | Filler | Silica | 3.0 | 3.0 | 3.0 |

| Total | | 100 | 100 | 100 |
|---|---|---|---|---|
| Blending | (i) Oil content (part by mass) excluding | 14.5 | 14.5 | 14.5 |
| ratio of | reactive polymer (vinyl dimethicone) | | | |
| catalyst | (ii) Platinum catalyst content (part by mass) | 0.024 | 0.024 | 0.024 |
| | Blending ratio (%) of catalyst to | 0.17 | 0.17 | 0.17 |
| | oil: {(ii) × 100}/(i) | | | |
| Evaluation | Cross-linking properties of film | A | A | A |
| | Appearance of film | A | A | A |
| | Abrasion resistance (number of repetition) | A(65) | A(64) | A(62) |
| | Emulsifiability | A | A | A |

¹⁾PEMULEN ™ EZ-4U POLYMER (Iwase Cosfa Co., Ltd.)
²⁾Divinyl disiloxane in the mixture is considered as an oil in the calculation of the blending ratio of catalysts to oils. The ratios of the ingredients in the mixture: 2% by mass of platinum, 4% by mass of divinyl disiloxane, and 94% by mass of vinyl dimethicone.

«Formulation Example of First Agent for Coating-Type Body Corrective Film Formation Agent»

Tables 9 to 12 below show formulation examples of first agents that can be used for coating-type body corrective film formation agents of the present disclosure, but the first agent for the coating-type body corrective film formation agent of the present invention is not limited to these examples. These formulations can be prepared by conventional methods.

TABLE 9

| | | | Form. | Form. |
|---|---|---|---|---|
| No. | Ingredient | Ingredient name | Ex. 14 | Ex. 15 |
| 1 | Second unsaturated | Divinyl dimethicone | 41.0 | 50.0 |
| | organopolysiloxane | (viscosity: 165,000 cSt) | | |
| 2 | Second hydride | Hydrogen dimethicone | 9.0 | 11.0 |
| | functionalized | (viscosity: 45 cSt) | | |
| | polysiloxane | | | |
| 3 | Filler | Silica silylate | 7.0 | 8.0 |
| 4 | Oil | Mixture of dimethicone | Balance | Balance |
| | | and trisiloxane | | |

| | | |
|---|---|---|
| Total | 100 | 100 |

TABLE 10

| | | | Form. | Form. | Form. |
|---|---|---|---|---|---|
| No. | Ingredient | Ingredient name | Ex. 16 | Ex. 17 | Ex. 18 |
| 1 | Second unsaturated | Divinyl dimethicone | 45.0 | 45.0 | 45.0 |
| | organopolysiloxane | (viscosity: 165,000 cSt) | | | |
| 2 | Second hydride | Hydrogen dimethicone | 10.0 | 10.0 | 10.0 |
| | functionalized | (viscosity: 45 cSt) | | | |
| | polysiloxane | | | | |
| 3 | Filler | Silica silylate | 7.5 | 7.5 | 7.5 |
| 4 | Oil | Cyclopentasiloxane | Balance | — | — |
| 5 | | Isododecane | — | Balance | — |
| 6 | | Crotonic acid/vinyl C8-12 | — | — | Balance |
| | | isoalkyl esters/VA/bis- | | | |
| | | vinyldimethicone | | | |
| | | crosspolymer and | | | |
| | | denatured alcohol | | | |

| | | | |
|---|---|---|---|
| Total | 100 | 100 | 100 |

TABLE 11

| | | | Form. | Form. | Form. |
|---|---|---|---|---|---|
| No. | Ingredient | Ingredient name | Ex. 19 | Ex. 20 | Ex. 21 |
| 1 | Second unsaturated | Divinyl dimethicone | 42.0 | 45.0 | 45.0 |
| | organopolysiloxane | (viscosity: 165,000 cSt) | | | |
| 2 | Second hydride | Hydrogen dimethicone | 10.0 | 10.0 | 10.0 |
| | functionalized | (viscosity: 50 cSt) | | | |
| | polysiloxane | | | | |
| 3 | Filler | Carbon | 10.0 | — | — |
| 4 | | Iron oxide | — | 10.0 | — |
| 5 | | Titanium oxide | — | — | 10.0 |
| 6 | Oil | Dimethicone | Balance | Balance | Balance |
| | | (viscosity: 1.5 cSt) | | | |

| | | | |
|---|---|---|---|
| Total | 100 | 100 | 100 |

TABLE 12

| | | | Form. | Form. | Form. |
|---|---|---|---|---|---|
| No. | Ingredient | Ingredient name | Ex. 22 | Ex. 23 | Ex. 24 |
| 1 | Second unsaturated | divinyl dimethicone | 25.0 | 20.0 | 20.0 |
| | organopolysiloxane | (viscosity: 165,000 cSt) | | | |
| 2 | Second hydride | Hydrogen dimethicone | 10.0 | 5.0 | 10.0 |
| | functionalized | (viscosity: 45 cSt) | | | |
| | polysiloxane | | | | |
| 3 | Water | Ion exchanged water | 40.0 | 40.0 | 35.0 |
| 4 | Salt | Sodium chloride | 1.0 | 1.0 | 5.0 |
| 5 | Polyhydric alcohol | 1,3-butylene glycol | 1.0 | 1.0 | 1.0 |
| 6 | Preservative | Phenoxyethanol | 0.5 | 0.5 | 0.5 |
| 7 | Oil | Dimethicone and | Balance | Balance | Balance |
| | | trisiloxane | | | |

| | | | |
|---|---|---|---|
| Total | 100 | 100 | 100 |

### REFERENCE SIGNS LIST

10, 20, and 30 skin
12, 22, and 32 first agent layer
14, 24, and 34 second agent layer
16, 26, and 36 catalyst
17, 27, and 37 oil
18 and 38 aqueous phase

1. An oil-in-water composition comprising:
a dispersion medium containing water, and
oil droplets dispersed in the dispersion medium,
wherein the oil droplets comprise an oil, and a catalyst that serves as a cross-linking ingredient,
wherein the oil-in-water composition is used as a second agent for a coating-type body corrective film formation agent comprising: a first agent comprising a cross-linking reactive ingredient that constitutes a body corrective film; and a second agent comprising a cross-linking ingredient for cross-linking the cross-linking reactive ingredient.

2. The composition according to claim 1, wherein the oil comprises a first unsaturated organopolysiloxane or a first hydride functionalized polysiloxane.

3. The composition according to claim 1, wherein the blending ratio of the catalyst to the oil is 0.060% or more, provided that, when the oil contains the first unsaturated organopolysiloxane or the first hydride functionalized polysiloxane, the blending ratio is calculated for the oil excluding the first unsaturated organopolysiloxane and the first hydride functionalized polysiloxane.

4. The composition according to claim 1, comprising at least one emulsifier selected from the group consisting of a hydrocarbon surfactant, a silicone surfactant, and an amphiphilic powder.

5. The composition according to claim 4, wherein the blending amount of the emulsifier is 5% by mass or less with respect to the total amount of the oil-in-water composition.

6. The composition according to claim 1, comprising a high-molecular-weight emulsifier.

7. The composition according to claim 6, wherein the blending amount of the high-molecular-weight emulsifier is 2% by mass or less with respect to the total amount of the oil-in-water composition.

8. The composition according to claim 1, wherein the catalyst is at least one selected from the group consisting of a platinum carbonyl cyclovinylmethylsiloxane complex, a platinum divinyltetramethyldisiloxane complex, a platinum cyclovinylmethylsiloxane complex, and a platinum octanaldehyde/octanol complex.

9. A coating-type body corrective film formation agent comprising a first agent and a second agent,
wherein the first agent comprises at least one selected from the group consisting of a second unsaturated organopolysiloxane and a second hydride functionalized polysiloxane,
wherein the second agent is an oil-in-water composition according to claim 1,
wherein when the first agent only comprises the second unsaturated organopolysiloxane out of the second unsaturated organopolysiloxane and the second hydride functionalized polysiloxane, the second agent comprises the first hydride functionalized polysiloxane, and
wherein when the first agent only comprises the second hydride functionalized polysiloxane out of the second unsaturated organopolysiloxane and the second hydride functionalized polysiloxane, the second agent comprises the first unsaturated organopolysiloxane.

10. The formation agent according to claim 9, wherein the first unsaturated organopolysiloxane and the second unsaturated organopolysiloxane are at least one selected from the group consisting of organopolysiloxanes containing a vinyl group, vinyl-terminated organopolysiloxanes, and organopolysiloxanes containing a vinylated branched chain.

11. The formation agent according to claim 9, wherein the first hydride functionalized polysiloxane and the second hydride functionalized polysiloxane are non-terminally and/or terminally hydroxylated organopolysiloxanes.

12. The formation agent according to claim 9, wherein at least one of the first agent and the second agent comprises at least one selected from the group consisting of a fiber, a pigment, a dye, a thickener, a UV absorber, and a filler.

13. A kit comprising the first agent and the second agent according to claim 9, wherein the first agent and the second agent are contained in separate containers, or contained in separate compartments of a container having two or more compartments.

14. A method of using the formation agent according to claim 9, comprising:
applying the first agent to the surface of a body to form a first agent layer, and then applying the second agent on the first agent layer to allow cross-linking, to form a body corrective film;
applying the second agent to the surface of a body to form a second agent layer, and then applying the first agent on the second agent layer to allow cross-linking, to form a body corrective film; or
mixing the first agent and the second agent to prepare a mixture, and then applying the mixture to the surface of a body to allow cross-linking, to form a body corrective film.

15. The method of use according to claim 14, comprising:
applying a cosmetic to the surface of the body before the application of the first agent, the second agent, or the mixture to the surface of the body;
applying the first agent to the surface of the body to form the first agent layer, applying a cosmetic to the first agent layer, and then applying the second agent to cover the cosmetic;
applying the second agent to the surface of the body to form the second agent layer, applying a cosmetic to the second agent layer, and then applying the first agent to cover the cosmetic; or
forming the body corrective film, and then applying a cosmetic to the film.
